(19) United States
(12) Patent Application Publication
(10) Pub. No.: US 2004/0138188 A1
(43) Pub. Date: Jul. 15, 2004

(54) Use of TGF-beta inhibitors to counteract pathologic changes in the level or function of steroid/thyroid receptors

(76) Inventors: Linda S. Higgins, Palo Alto, CA (US); Ann M. Kapoun, Mountian View, CA (US); David Y. Liu, Palo Alto, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/719,082

(22) Filed: Nov. 20, 2003

Related U.S. Application Data
(60) Provisional application No. 60/428,860, filed on Nov. 22, 2002.

(51) Int. Cl. A61K 31/56

(52) U.S. Cl. 514/169

(57) ABSTRACT

The invention concerns the use of TGF-β inhibitors to counteract a pathologic change in the expression level, activity and/or signaling of a receptor of the steroid-thyroid hormone receptor superfamily. In particular, the invention concerns a method for counteracting a pathologic change in a signal-transduction pathway involving a member of the steroid/thyroid hormone super-family, comprising administering to a mammalian subject in need an effective amount of a compound capable of inhibiting TGF-β signaling through a TGF-β receptor.

### CROSS-REFERENCE TO RELATED APPLICATIONS

This is a non-provisional application filed under 37 C.F.R. 1.53(b), claiming priority under 35 U.S.C. § 119(e) to Provisional Application Serial No. 60/428,860, filed on Nov. 22, 2002.

### BACKGROUND OF THE INVENTION

1. Field of the Invention

The present invention concerns the use of TGF-β inhibitors to counteract a pathologic change in the expression level, activity and/or signaling of a receptor of the steroid-thyroid hormone receptor superfamily.

2. Description of the Related Art

The Steroid/Thyroid Hormone Receptor Super-Family

The steroid and thyroid hormone receptor super-family of proteins includes receptors for steroid hormones, thyroid hormones, Vitamin D, and retinoic acid (Vitamin A). When these receptors bind their respective ligands, they undergo a conformational change resulting in activation that enables the receptors to recognize and bind to specific nucleotide sequences, referred to as hormone responsive elements (HREs). When ligand-receptor complexes interact with DNA, they alter the transcriptional level of the associated gene. All members of the steroid/thyroid hormone receptor super-family share, in an N-terminal to C-terminal direction, a transcriptional regulatory domain, a zinc-finger domain needed for DNA binding, and a domain responsible for binding a particular hormone and for dimerization (ligand binding a dimerization domain).

Representative members of the steroid receptor family are the glucocorticoid and progesterone receptors. The family further includes receptors for other steroid hormones, like aldosterone, estrogens, thyroid hormones, etc. Corticosteroids are effective anti-inflammatory drugs, widely used to treat various inflammatory diseases, including, for example, rheumatoid arthritis (RA), inflammatory bowel disease (IBD), dermatitis, and asthma, just to mention a few. As corticosteroids exert their activity by interaction with their receptors, down-regulation of their receptors decreases the efficacy of corticosteroid therapy.

Retinoic acid receptors (RARs) include at least three sub-types RARα, RARβ, and RARγ. These receptor sub-types exhibit highest affinity for all-trans-retinoic acid (all-trans-RA). The family also includes the so called retinoid X receptors (RXRs), which again have three subtypes, RXRα, RXRβ, and RXRγ, exhibiting highest affinity for 9-cis-retinoic acid (9-cis-RA). Retinoids play an important role in normal development, physiology and cancer prevention. Various retinoids have been shown to have anti-tumor activity. Thus, all-trans retinoic acid (ATRA) has been used successfully in cancer therapy, for example for the treatment of breast cancer and acute promyelocytic leukemia. As retinoids exert their biological activities by interaction with their respective receptors, down-regulation of the retonic acid receptors reduces the efficacy of retinoid treatment, e.g. in cancer therapy.

Thyroid hormone receptors are classified into α and β families. Currently, thyroid receptors include TRα-1, TRα-2, TRβ-1, and TRβ-2. The expression pattern of the thyroid receptors varies by tissue and by developmental stage. While the TRα-1, TRα-2, and TRβ-1 isoforms are widely expressed in almost all tissues, the TRβ-2 isoform is expressed almost exclusively in the hypothalamus, the anteriod pituitary and the developing ear. Upregulation of the latter receptor is believed to be important to the effect of thyroid hormones on the development of the fetal and neonatal brain. Thyroid hormone therapy is often used in the treatment of underactive thyroid gland to raise abnormally low levels of thyroid hormones. Thyroid hormones are also administered to control the growth of thyroid gland, which may be enlarged (as in the case of goiter), or contain nudules. Down-regulation of the thyroid hormone receptors reduces the efficacy of thyroid hormone treatment. A pathologic increase in the level of a thyroid receptor may be associated, for example, with Grave's disease.

Transforming Growth Factor-Beta

Transforming growth factor-beta (TGF-β) denotes a family of proteins, TGF-β1, TGF-β2, and TGF-β3, which are pleiotropic modulators of cell growth and differentiation, embryonic and bone development, extracellular matrix formation, hematopoiesis, immune and inflammatory responses (Roberts and Sporn Handbook of Experimental Pharmacology (1990) 95:419-58; Massague et al. Ann Rev Cell Biol (1990) 6:597-646). Other members of this super-family include activin, inhibin, bone morphogenic protein, and Mullerian inhibiting substance. TGF-β initiates intracellular signaling pathways leading ultimately to the expression of genes that regulate the cell cycle, control proliferative responses, or relate to extracellular matrix proteins that mediate outside-in cell signaling, cell adhesion, migration and intercellular communication.

TGF-β, including TGF-β1, -β2 and -β3, exerts its biological activities through a receptor system including the type I and type II single transmembrane TGF-β receptors (also referred to as receptor subunits) with intracellular serine-threonine kinase domains, that signal through the Smad family of transcriptional regulators. Binding of TGF-β to the extracellular domain of the type II receptor induces phosphorylation and activation of the type I receptor (TGFβ-R1) by the type II receptor (TGFβ-R2). The activated TGFβ-R1 phosphorylates a receptor-associated co-transcription factor Smad2/Smad3, thereby releasing it into the cytoplasm, where it binds to Smad4. The Smad complex translocates into the nucleus, associates with a DNA-binding cofactor, such as Fast-1, binds to enhancer regions of specific genes, and activates transcription. The expression of these genes leads to the synthesis of cell cycle regulators that control proliferative responses or extracellular matrix proteins that mediate outside-in cell signaling, cell adhesion, migration, and intracellular communication. Other signaling pathways like the MAP kinase-ERK cascade are also activated by TGF-β signaling. For review, see, e.g. Whitman, Genes Dev. 12:2445-62 (1998); and Miyazono et al., Adv. Immunol. 75:111-57 (2000), which are expressly incorporated herein by reference. Further information about the TGF-β signaling pathway can be found, for example, in the following publications: Attisano et al., “Signal transduction by the TGF-β super-family” Science 296:1646-7 (2002); Bottinger and Bitzer, “TGF-β signaling in renal disease” Am. Soc. Nephrol. 13:2600-2610 (2002); Topper, J. N., “TGF-β in the cardiovascular system: molecular mechanisms of a context-specific growth factor” Trends Cardiovasc. Med. 10:132-7 (2000), review; Itoh et al., “Signaling of transforming growth factor-β family” Eur. J. Biochem. 267:6954-67 (2000), review.

### SUMMARY OF THE INVENTION

In one aspect, the invention concerns a method for counteracting a pathologic change in a signal-transduction pathway involving a member of the steroid/thyroid hormone super-family, comprising administering to a mammalian subject in need an effective amount of a compound capable of inhibiting TGF-β signaling through a TGF-β receptor.

In a particular embodiment, the pathologic change is down-regulation or up-regulation of a steroid hormone receptor, such as a glucocortocid receptor. In a specific embodiment, down-regulation or up-regulation of the steroid hormone receptor involves TGF-β. In another specific embodiment, the down- or up-regulation is induced by TGF-β.

In another embodiment, the pathologic change is down-regulation or up-regulation of a thyroid hormone receptor. In a specific embodiment, down-regulation or up-regulation of the thyroid hormone receptor involves TGF-β. In another particular embodiment, down- or up-regulation is induced by TGF-β.

In a further embodiment, the pathologic change is down-regulation or up-regulation of a retinoic acid receptor. In a specific embodiment, down-regulation or up-regulation of the retinoic acid receptor involves TGF-β. In another specific embodiment, the down- or up-regulation is induced by TGF-β.

In another embodiment, the pathologic change is a pathologic change in the activity and/or signaling of a member of the steroid/thyroid hormone super-family, such as a steroid, thyroid, or retinoic acid receptor.

In all embodiments, a preferred TGF-β receptor is a TGFβ-R1 kinase. In a particular embodiment, the compound capable of inhibiting TGF-β signaling through a TGF-β receptor binds to a TGFβ-R1 kinase. In another particular embodiment, the compound may additionally bind to at least one further receptor kinase, such as an activin receptor (Alk4).

The molecules used in practicing the present invention are preferably non-peptide small molecules, e.g. small organic molecules.

A preferred group of the small organic molecules of the present invention is represented by the formula (1)

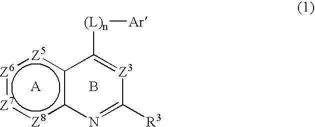

or the pharmaceutically acceptable salts thereof

wherein R3 is a noninterfering substituent;

each Z is CR2 or N, wherein no more than two Z positions in ring A are N, and wherein two adjacent Z positions in ring A cannot be N;

each R2 is independently a noninterfering substituent;

L is a linker;

n is 0 or 1; and

Ar′ is the residue of a cyclic aliphatic, cyclic heteroaliphatic, aromatic or heteroaromatic moiety optionally substituted with 1-3 noninterfering substituents.

Another group of the small organic molecules herein are represented by the

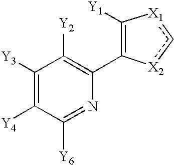

formula (2)

wherein Y₁ is phenyl or naphthyl optionally substituted with one or more substituents selected from halo, alkoxy(1-6 C), alkylthio(1-6 C), alkyl(1-6 C), haloalkyl (1-6C), —O—(CH₂)ₘ-Ph, —S—(CH₂)ₘ-Ph, cyano, phenyl, and CO₂R, wherein R is hydrogen or alkyl(1-6 C), and m is 0-3; or phenyl fused with a 5- or 7-membered aromatic or non-aromatic ring wherein said ring contains up to three heteroatoms, independently selected from N, O, and

Y₂, Y₃, Y₄, and Y₅ independently represent hydrogen, alkyl(1-6C), alkoxy(1-6 C), haloalkyl(1-6 C), halo, NH2, NH-alkyl(1-6C), or NH(CH₂)ₙ-Ph wherein n is 0 -3; or an adjacent pair of Y₂, Y₃, Y₄, and Y₅ form a fused 6-membered aromatic ring optionally containing up to 2 nitrogen atoms, said ring being optionally substituted by one or more substituents independently selected from alkyl(1-6 C), alkoxy(a-6 C), haloalkyl(1-6 C), halo, NH₂, NH-alkyl(1-6 C), or NH(CH₂)ₙ-Ph, wherein n is 0-3, and the remainder of Y₂, Y₃, Y₄, and Y₅ represent hydrogen, alkyl(1-6 C), alkoxy(1-6C), haloalkyl(1-6 C), halo, NH₂, NH-alkyl(1-6 C), or NH(CH₂)ₙ-Ph wherein n is 0-3; and

one of X₁ and X₂ is N and the other is NR₆, wherein R₆ is hydrogen or alkyl(1-6 C).

A further group of the small organic molecules herein is represented by the formula (3)

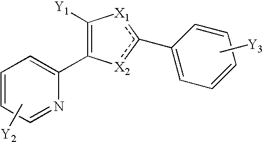

wherein Y₁ is naphthyl, anthracenyl, or phenyl optionally substituted with one or more substituents selected from the group consisting of halo, alkoxy(1-6 C), alkylthio(1-6 C), alkyl(1-6 C), —O—(CH₂)-Ph, —S—(CH₂)ₙ-Ph, cyano, phenyl, and CO₂R,

wherein R is hydrogen or alkyl(1-6 C), and n is 0, 1, 2, or 3; or Y₁ represents phenyl fused with an aromatic or non-aromatic cyclic ring of 5-7 members wherein said cyclic ring optionally contains up to two heteroatoms, independently selected from N, O, and S;

Y₂ is H, NH(CH₂)ₙ-Ph or NH-alkyl(1-6 C), wherein n is 0, 1, 2, or 3; Y₃ is CO₂H, CONH₂, CN, NO₂, alkylthio(1-6 C), —SO₂-alkyl(C1-6), alkoxy(C1-6), SONH₂, CONHOH, NH₂, CHO, CH₂NH₂, or CO₂R, wherein R is hydrogen or alkyl(1-6 C);

one of X₁ and X₂ is N or CR′, and other is NR′ or CHR′ wherein R′ is hydrogen, OH, alkyl(C-16), or cycloalkyl(C3-7); or when one of X₁ and X₂ is N or CR′ then the other may be S or O.

Yet another group of the small organic molecules herein is represented by the following formula (4)

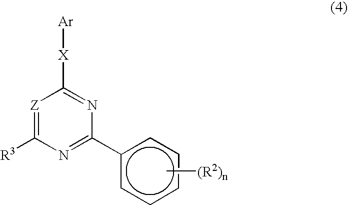

and the pharmaceutically acceptable salts and prodrug forms thereof; wherein

Ar represents an optionally substituted aromatic or optionally substituted heteroaromatic moiety containing 5-12 ring members wherein said heteroaromatic moiety contains one or more O, S, and/or N with a proviso that the optionally substituted Ar is not

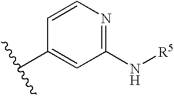

wherein R⁵ is H, alkyl (1-6C), alkenyl (2-6C), alkynyl (2-6C), an aromatic or heteroaromatic moiety containing 5-11 ring members;

X is NR¹, O, or S;

R¹ is H, alkyl (1-8C), alkenyl (2-8C), or alkynyl (2-8C);

Z represents N or CR⁴;

each of R³ and R⁴ is independently H, or a non-interfering substituent;

each R² is independently a non-interfering substituent; and

n is 0, 1, 2, 3, 4, or 5.

In one embodiment, if n>2, and the R2's are adjacent, they can be joined together to form a 5 to 7 membered non-aromatic, heteroaromatic, or aromatic ring containing 1 to 3 heteroatoms where each heteroatom can independently be O, N, or S.

Further small organic compounds within the scope herein are represented by formula (5)

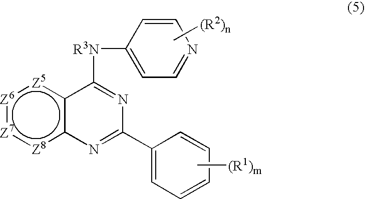

or the pharmaceutically acceptable salts thereof;

wherein each of Z⁵, Z⁶, Z⁷ and Z⁸ is N or CH and wherein one or two Z⁵, Z⁶, Z⁷ and Z⁸ are N and wherein two adjacent Z positions cannot be N;

wherein m and n are each independently 0-3;

wherein two adjacent R¹ groups may be joined to form an aliphatic heterocyclic ring of 5-6 members;

wherein R² is a noninterfering substituent; and

wherein R³ is H or CH₃.

### BRIEF DESCRIPTION OF THE DRAWINGS

FIG. 1 illustrates that TGFβ1-induced down-regulation of the glucocorticoid receptor is reversed by two representative TGFβ-R1 inhibitor Compound Nos. 74 and 81 of the invention in rat lung fibroblasts and normal rat kidney.

FIG. 2 shows that TGFβ1-induced down-regulation of steroid/thyroid receptors are reversed by TGFβ-R1 inhibitor compound No. 79 in human lung fibroblast cells.

### DETAILED DESCRIPTION OF THE PREFERRED EMBODIMENT

A. Definitions

Unless defined otherwise, technical and scientific terms used herein have the same meaning as commonly understood by one of ordinary skill in the art to which this invention belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). For purposes of the present invention, the following terms are defined below.

The term “TGF-β” is used herein to include native sequence TGF-β1, TGF-β2 and TGF-β3 of all mammalian species, including any naturally occurring variants of the TGF-β polypeptides.

The term “counteracting a pathologic change” is used in the broadest sense, and refers to any action that prevents, circumvents, reverses, compensates for, slows down, blocks, or limits the pathologic change, regardless the underlying mechanism. Pathologic changes specifically include, without limitation, changes in the expression level, activity and/or signaling of a receptor of the steroid/thyroid receptor superfamily.

Down-regulation of a receptor “involves TGF-β” if TGF-β plays any role whatsoever, either directly or indirectly, in such down-regulation. The term includes, but is not limited to, down-regulation caused by direct exposure of the receptor to endogenous or exogenous TGF-β.

Similarly, up-regulation of a receptor “involves TGF-β” if TGF-β plays any role whatsoever, either directly or indirectly, in such up-regulation. The term includes, but is not limited to, up-regulation caused by direct exposure of the receptor to endogenous or exogenous TGF-β.

As used herein, the term “inflammatory disease” or “inflammatory disorder” refers to pathological states resulting in inflammation, typically caused by neutrophil chemotaxis. Examples of such disorders include inflammatory skin diseases including psoriasis and atopic dermatitis; systemic scleroderma and sclerosis; responses associated with inflammatory bowel disease (IBD) (such as Crohn's disease and ulcerative colitis); ischemic reperfusion disorders including surgical tissue reperfusion injury, myocardial ischemic conditions such as myocardial infarction, cardiac arrest, reperfusion after cardiac surgery and constriction after percutaneous transluminal coronary angioplasty, stroke, and abdominal aortic aneurysms; cerebral edema secondary to stroke; cranial trauma, hypovolemic shock; asphyxia; adult respiratory distress syndrome; acute-lung injury; Behcet's Disease; dermatomyositis; polymyositis; multiple sclerosis (MS); dermatitis; meningitis; encephalitis; uveitis; osteoarthritis; lupus nephritis; autoimmune diseases such as rheumatoid arthritis (RA), Sjorgen's syndrome, vasculitis; diseases involving leukocyte diapedesis; central nervous system (CNS) inflammatory disorder, multiple organ injury syndrome secondary to septicaemia or trauma; alcoholic hepatitis; bacterial pneumonia; antigen-antibody complex mediated diseases including glomerulonephritis; sepsis; sarcoidosis; immunopathologic responses to tissue/organ transplantation; inflammations of the lung, including pleurisy, alveolitis, vasculitis, pneumonia, chronic bronchitis, bronchiectasis, diffuse panbronchiolitis, hypersensitivity pneumonitis, idiopathic pulmonary fibrosis (IPF), and cystic fibrosis; etc. The preferred indications include, without limitation, chronic inflammation, autoimmune diabetes, rheumatoid arthritis (RA), rheumatoid spondylitis, gouty arthritis and other arthritic conditions, multiple sclerosis (MS), asthma, systhemic lupus erythrematosus, adult respiratory distress syndrome, Behcet's disease, psoriasis, chronic pulmonary inflammatory disease, graft versus host reaction, Crohn's Disease, ulcerative colitis, inflammatory bowel disease (IBD), Alzheimer's disease, and pyresis, along with any disease or disorder that relates to inflammation and related disorders.

A “biological activity mediated by the TGFβ-R1 kinase receptor,” or “biological activity mediated by a TGFβ-R1 receptor” can be any activity associated with the activation of TGFβ-R1 and downsteam intracellular signaling events, such as the phosphorylation of Smad2/Smad3, or any signaling effect occurring in the Smad-independent signaling arm of the TGF-β signal transduction cascad, including, for example, p38 and ras.

The term “treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

The “pathology” of a disease or condition includes all phenomena that compromise the well-being of the patient.

The term “TGF-β inhibitor” as used herein refers to a molecule having the ability to inhibit a biological function of a native TGF-β molecule mediated by a TGF-βreceptor kinase, such as the TGFβ-R1 or TGF-R2 receptor, by interacting with a TGF-βreceptor kinase. Accordingly, the term “inhibitor” is defined in the context of the biological role of TGF-β and its receptors. While the inhibitors herein are characterized by their ability to interact with a TGF-β receptor kinase and thereby inhibiting TGF-β biological function, they might additionally interact with other members in the TGF-β signal transduction pathway or members shared by the TGF-β signal transduction pathway and another pathway. Thus, the term “TGF-β inhibitor” specifically includes molecules capable of interacting with and inhibiting the biological function of two or more receptor kinases, including, without limitation, an activin receptor kinase, e.g. Alk4, and/or a MAP kinase.

The term “interact” with reference to an inhibitor and a receptor includes binding of the inhibitor to the receptor as well as indirect interaction, which does not involve binding. The binding to a receptor can, for example, be specific or preferential.

The terms “specifically binding,” “binds specifically,” “specific binding,” and grammatical variants thereof, are used to refer to binding to a unique epitope within a target molecule, such as a TGFβ receptor, e.g. the type I TGF-β receptor (TGFβ-R1). The binding must occur with an affinity to effectively inhibit TGF-β signaling through the receptor, e.g. TGFβ-R1.

The terms “preferentially binding,” binds preferentially,” “preferential binding,” and grammatical variants thereof, as used herein means that binding to one target is significantly greater than binding to any other binding partner. The binding affinity to the preferentially bound target is generally at least about two-fold, more preferably at least about five-fold, even more preferably at least about ten-fold greater than the binding affinity to any other binding partner.

The term “preferentially inhibit” as used herein means that the inhibitory effect on the target that is “preferentially inhibited” is significantly greater than on any other target. Thus, for example, in the context of preferential inhibition of TGF-β-R1 kinase relative to the p38 kinase, the term means that the inhibitor inhibits biological activities mediated by the TGF-β-R1 kinase significantly more than biological activities mediated by the p38 kinase. The difference in the degree of inhibition, in favor of the preferentially inhibited receptor, generally is at least about two-fold, more preferably at least about five-fold, even more preferably at least about ten-fold.

The term “mammal” for purposes of treatment refers to any animal classified as a mammal, including humans, domestic and farm animals, and zoo, sports, or pet animals, such as dogs, cats, cattle, horses, sheep, pigs, goats, rabbits, etc. Preferably, the mammal is human.

Administration “in combination with” one or more further therapeutic agents includes simultaneous (concurrent) and consecutive administration in any order.

A “therapeutically effective amount”, in the context of the present invention refers to an amount capable of counteracting a pathologic change in a β-adrenergic pathway, as defined above. In reference to the treatment of a disease or condition, the term “therapeutically effective amount” refers to an amount capable of invoking one or more of the following effects: (1) prevention of the disease or condition; (2) inhibition (i.e., reduction, slowing down or complete stopping) of the development or progression of the disease or condition; (3) inhibition (i.e., reduction, slowing down or complete stopping) of consequences of or complications resulting from such disease or condition; and (4) relief, to some extent, of one or more symptoms associated with such disease or condition, or symptoms of consequences of or complications resulting from such disease and/or condition.

As used herein, a “noninterfering substituent” is a substituent which leaves the ability of the compound of formula (1) to inhibit TGF-β activity qualitatively intact. Thus, the substituent may alter the degree of inhibition. However, as long as the compound of formula (1) retains the ability to inhibit TGF-β activity, the substituent will be classified as “noninterfering.”

As used herein, “hydrocarbyl residue” refers to a residue which contains only carbon and hydrogen. The residue may be aliphatic or aromatic, straight-chain, cyclic, branched, saturated or unsaturated. The hydrocarbyl residue, when indicated, may contain heteroatoms over and above the carbon and hydrogen members of the substituent residue. Thus, when specifically noted as containing such heteroatoms, the hydrocarbyl residue may also contain carbonyl groups, amino groups, hydroxyl groups and the like, or contain heteroatoms within the “backbone” of the hydrocarbyl residue.

As used herein, the term “alkyl,” “alkenyl” and “alkynyl” include straight- and branched-chain and cyclic monovalent substituents. Examples include methyl, ethyl, isobutyl, cyclohexyl, cyclopentylethyl, 2-propenyl, 3-butynyl, and the like. Typically, the alkyl, alkenyl and alkynyl substituents contain 1-10C (alkyl) or 2-10C (alkenyl or alkynyl). Preferably they contain 1-6C (alkyl) or 2-6C (alkenyl or alkynyl). Heteroalkyl, heteroalkenyl and heteroalkynyl are similarly defined but may contain 1-2 O, S or N heteroatoms or combinations thereof within the backbone residue.

As used herein, “acyl” encompasses the definitions of alkyl, alkenyl, alkynyl and the related hetero-forms which are coupled to an additional residue through a carbonyl group.

“Aromatic” moiety refers to a monocyclic or fused bicyclic moiety such as phenyl or naphthyl; “heteroaromatic” also refers to monocyclic or fused bicyclic ring systems containing one ore more heteroatoms selected from O, S and N. The inclusion of a heteroatom permits inclusion of 5-membered rings as well as 6-membered rings. Thus, typical aromatic systems include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, imidazolyl and the like. Any monocyclic or fused ring bicyclic system which has the characteristics of aromaticity in terms of electron distribution throughout the ring system is included in this definition. Typically, the ring systems contain 5-12 ring member atoms.

Similarly, “arylalkyl” and “heteroalkyl” refer to aromatic and heteroaromatic systems which are coupled to another residue through a carbon chain, including substituted or unsubstituted, saturated or unsaturated, carbon chains, typically of 1-6C. These carbon chains may also include a carbonyl group, thus making them able to provide substituents as an acyl moiety.

B. Modes of Carrying Out the Invention

The present invention is based on the surprising discovery that compounds capable of inhibiting TGFβ signaling through a TGFβ receptor can counteract pathologic changes in pathways involving signaling through members of the steroid/thyroid receptor super-family. In particular, the invention is based on the discovery that TGFβ-induced down-regulation of steroid/thyroid receptors can be reversed by compounds capable of inhibiting TGFβ signaling through a TGFβ receptor.

As discussed before, steroids, in particularly corticosteroids, are used as antiinflammatory agents or immunsuppressants in the treatment of a wide range of diseases, including various inflammatory diseases and conditions, autoimmune diseases and in transplant surgery.

Inflammatory diseases typically treated with corticosteroids include, without limitation, inflammatory skin diseases including psoriasis and atopic dermatitis; systemic scleroderma and sclerosis; responses associated with inflammatory bowel disease (IBD) (such as Crohn's disease and ulcerative colitis); ischemic reperfusion disorders including surgical tissue reperfusion injury, myocardial ischemic conditions such as myocardial infarction, cardiac arrest, reperfusion after cardiac surgery and constriction after percutaneous transluminal coronary angioplasty, stroke, and abdominal aortic aneurysms; cerebral edema secondary to stroke; cranial trauma, hypovolemic shock; asphyxia; adult respiratory distress syndrome; acute-lung injury; Behcet's Disease; dermatomyositis; polymyositis; multiple sclerosis (MS); dermatitis; meningitis; encephalitis; uveitis; osteoarthritis; lupus nephritis; autoimmune diseases such as rheumatoid arthritis (RA), Sjorgen's syndrome, vasculitis; diseases involving leukocyte diapedesis; central nervous system (CNS) inflammatory disorder, multiple organ injury syndrome secondary to septicaemia or trauma; alcoholic hepatitis; bacterial pneumonia; antigen-antibody complex mediated diseases including glomerulonephritis; sepsis; sarcoidosis; immunopathologic responses to tissue/organ transplantation; inflammations of the lung, including pleurisy, alveolitis, vasculitis, pneumonia, chronic bronchitis, bronchiectasis, diffuse panbronchiolitis, hypersensitivity pneumonitis, idiopathic pulmonary fibrosis (IPF), and cystic fibrosis; etc. For example, steroids, e.g. prednisolone and methylprednisolone, are often used to treat acute attacks of multiple sclerosis. Steroids are also prescribed for the treatment and management of various respiratory diseases involving inflammation, such as asthma. Administration of corticosteroids, e.g. dexamethasone, has been proposed for the treatment of Huntington chorea.

The most common use of thyroid hormones is in hypothyroidism and in the treatment of diseases associated with abnormal growth or development of the thyroid gland, e.g. goiter.

Retinoid treatment is often used in dermatology to reverse or reduce skin abnormalities, such as in the treatment of acne. Retinoids, such as Vitamin A, are also known for their anti-cancer activities, which are believed to be associated with the anti-oxidant properties of these compounds. Retinoids have been described as promising agents for the tratment of cancer, in particular breast cancer and acute promyelocytic leukemia. The effect of retinoids is thought to result from modulation of gene activity by at least two distinct class of nuclear receptors, the retinoic acid receptors (RARs) and the retinoid X receptors (RXRs). As discussed earlier, these receptors exist as major subtypes α, β, and γ. It has been reported that retinoic acid (RA) is able to induce RARE in breast cancer cells and that this induction correlates with RA growth inhibitory effect. These observations suggest that RARE may be essential for the anti-growth effect of RA.

Since the listed hormones exert their activities through their respective receptors, down-regulation of the receptors, or any negative change in the activity of and/or signaling through these receptors may interfere with the efficacy of the therapeutic use of these hormones. The present invention provides a solution to this problem by teaching the administration of compounds that are capable of reversing the down-regulation of receptors of the steroid/thyroid hormone superfamily.

Similarly, increased levels of steroid or thyroid hormone receptors, or retinoic acid receptors may be pathological. For example, increased levels of a thyroid receptor may be associated with Grave's disease. The compounds of the present invention are also suitable for counteracting pathologic changes characterized by over-expression or a receptor of the steroid/thyroid hormone superfamily, especially when the over-expression is induced by TGF-β.

C. Compounds of the Invention

The compounds of the present invention are capable of inhibiting TGFβ signaling through a TGFβ receptor and, as a result, can counteract pathologic changes in the β-adrenergic signal transduction pathway. As discussed earlier, a TGF-β inhibitor, as defined for the purpose of the present invention, can be any molecule having the ability to inhibit a biological function of a native TGF-β molecule mediated by a TGF-β receptor kinase, such as the TGFβ-R1 or TGF-R2 receptor via interaction with a TGF-β receptor kinase. Although the inhibitors are characterized by their ability to interact with a TGF-β receptor kinase and thereby inhibiting TGF-β biological function, they might additionally interact with other members in the TGF-β signal transduction pathway or members shared by the TGF-β signal transduction pathway and another pathway. Thus, TGF-β inhibitors might interact with two or more receptor kinases.

As discussed earlier, the type 1 and type 2 TGF-β receptors are serine-threonine kinases that signal through the Smad family of transcriptional regulators. Binding of TGF-β induces phosphorylation and activation of TGFβ-R1 by the TGFβ-R2. The activated TGFβ-R1 phosphorylates Smad2 and Smad3, which bind to Smad4 to move into the nucleus and form transcription regulatory complexes. Other signaling pathways, such as the MAP kinase-ERK cascade are also activated by TGF-β signaling, and modulate Smad activation. The Smad proteins couple the activation of both the TGF-β and the activin receptors to nuclear transcription. Thus, the TGF-β inhibitors of the present invention may additionally interact with an activin receptor kinase, such as Alk4, and/or a MAP kinase.

The compounds of the present invention include, without limitation, polypeptides, including antibodies and antibody-like molecules, peptides, polynucleotides, antisense molecules, decoys, and non-peptide small organic molecules that are capable of inhibiting TGF-β signaling through a TGF-β receptor.

In a particular embodiment, the compounds of the present invention are small organic molecules (non-peptide small molecules), generally less than about 1,000 daltons in size. Preferred non-peptide small molecules have molecular weights of less than about 750 daltons, more preferably less than about 500 daltons, and even more preferably less than about 300 daltons.

In a preferred embodiment, the compounds of the invention are of the formula

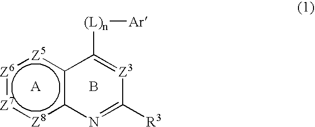

or the pharmaceutically acceptable salts thereof

wherein R³ is a noninterfering substituent; each Z is CR² or N, wherein no more than two Z positions in ring A are N, and wherein two adjacent Z positions in ring A cannot be N;

each R² is independently a noninterfering substituent;

L is a linker;

n is 0 or 1; and

Ar′ is the residue of a cyclic aliphatic, cyclic heteroaliphatic, aromatic or heteroaromatic moiety optionally substituted with 1-3 noninterfering substituents.

In a preferred embodiment, the small organic molecules herein are derivatives of quinazoline and related compounds containing mandatory substituents at positions corresponding to the 2- and 4-positions of quinazoline. In general, a quinazoline nucleus is preferred, although alternatives within the scope of the invention are also illustrated below. Preferred embodiments for Z³ are N and CH; preferred embodiments for Z⁵-Z⁸ are CR². However, each of Z⁵-Z⁸ can also be N, with the proviso noted above. Thus, with respect to the basic quinazoline type ring system, preferred embodiments include quinazoline per se, and embodiments wherein all of Z⁵-Z⁸ as well as Z³ are either N or CH. Also preferred are those embodiments wherein Z³ is N, and either Z⁵ or Z⁸ or both Z⁵ and Z⁸ are N and Z and Z⁷ are CH or CR². Where R² is other than H, it is preferred that CR² occur at positions 6 and/or 7. Thus, by way of example, quinazoline derivatives within the scope of the invention include compounds comprising a quinazoline nucleus, having an aromatic ring attached in position 2 as a non-interfering substituent (R³), which may be further substituted.

With respect to the substituent at the positions corresponding to the 4-position of quinazoline, LAr′, L is present or absent and is a linker which spaces the substituent Ar′ from ring B at a distance of 2-8 Å, preferably 2-6 Å, more preferably 2-4 Å. The distance is measured from the ring carbon in ring B to which one valence of L is attached to the atom of the Ar′ cyclic moiety to which the other valence of the linker is attached. The Ar′ moiety may also be coupled directly to ring B (i.e., when n is 0). Typical, but nonlimiting, embodiments of L are of the formula S(CR²₂)ₘ, —NR¹SO₂(CR²₂)ₗ, NR¹(CR²₂)ₘ, NR¹CO(CR²₂)ₗ, O(CR²₂)ₘ, OCO(CR²₂)ₗ, and

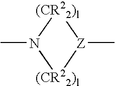

wherein Z is N or CH and wherein m is 0-4 and 1 is 0-3, preferably 1-3 and 1-2, respectively. L preferably provides —NR¹— coupled directly to ring B. A preferred embodiment of R′ is H, but R′ may also be acyl, alkyl, arylacyl or arylalkyl where the aryl moiety may be substituted by 1-3 groups such as alkyl, alkenyl, alkynyl, acyl, aryl, alkylaryl, aroyl, N-aryl, NH-alkylaryl, NH-aroyl, halo, OR, NR₂, SR, —SOR, —NRSOR, —NRSO₂R, —SO₂R, —OCOR, —NRCOR, —NRCONR₂, —NRCOOR, —OCONR₂, —RCO, —COOR, —SO₃R, —CONR₂, SO₂NR₂, CN, CF₃, and NO₂, wherein each R is independently H or alkyl (1-4C), preferably the substituents are alkyl (1-6C), OR, SR or NR₂ wherein R is H or lower alkyl (1-4C). More preferably, R¹ is H or alkyl (1-6C). Any aryl groups contained in the substituents may further be substituted by for example alkyl, alkenyl, alkynyl, halo, OR, NR₂, SR, —SOR, —SO₂R, —OCOR, —NRCOR, —NRCONR₂, —NRCOOR, —OCONR₂, —RCO, —COOR, SO₂R, NRSOR, NRSO₂R, —SO₃R, —CONR₂, SO₂NR₂, CN, CF₃, or NO₂, wherein each R is independently H or alkyl (1-4C).

Ar′ is aryl, heteroaryl, including 6-5 fused heteroaryl, cycloaliphatic or cycloheteroaliphatic. Preferably Ar′ is phenyl, 2-, 3- or 4-pyridyl, indolyl, 2- or 4-pyrimidyl, benzimidazolyl, indolyl, preferably each optionally substituted with a group selected from the group consisting of optionally substituted alkyl, alkenyl, alkynyl, aryl, N-aryl, NH-aroyl, halo, OR, NR₂, SR, —OOCR, —NROCR, RCO, —COOR, —CONR₂, SO₂NR₂, CN, CF₃, and NO₂, wherein each R is independently H or alkyl (1-4C).

Ar′ is more preferably indolyl, 6-pyrimidyl, 3- or 4-pyridyl, or optionally substituted phenyl.

For embodiments wherein Ar′ is optionally substituted phenyl, substituents include, without limitation, alkyl, alkenyl, alkynyl, aryl, alkylaryl, aroyl, N-aryl, NH-alkylaryl, NH-aroyl, halo, OR, NR₂, SR, —SOR, —SO₂R, —OCOR, —NRCOR, —NRCONR₂, —NRCOOR, —OCONR₂, RCO, —COOR, —SO₃R, —CONR₂, SO₂NR₂, CN, CF₃, and NO₂, wherein each R is independently H or alkyl (1-4C). Preferred substituents include halo, OR, SR, and NR₂ wherein R is H or methyl or ethyl. These substituents may occupy all five positions of the phenyl ring, preferably 1-2 positions, preferably one position. Embodiments of Ar′ include substituted or unsubstituted phenyl, 2-, 3-, or 4-pyridyl, 2-, 4- or 6-pyrimidyl, indolyl, isoquinolyl, quinolyl, benzimidazolyl, benzotriazolyl, benzothiazolyl, benzofuranyl, pyridyl, thienyl, furyl, pyrrolyl, thiazolyl, oxazolyl, imidazolyl, and morpholinyl. Particularly preferred as an embodiment of Ar′ is 3- or 4-pyridyl, especially 4-pyridyl in unsubstituted form.

Any of the aryl moieties, especially the phenyl moieties, may also comprise two substituents which, when taken together, form a 5-7 membered carbocyclic or heterocyclic aliphatic ring.

Thus, preferred embodiments of the substituents at the position of ring B corresponding to 4-position of the quinazoline include 2-(4-pyridyl)ethylamino; 4-pyridylamino; 3-pyridylamino; 2-pyridylamino; 4-indolylamino; 5-indolylamino; 3-methoxyanilinyl; 2-(2,5-difluorophenyl)ethylamino-, and the like.

R³ is generally a hydrocarbyl residue (1-20C) containing 0-5 heteroatoms selected from O, S and N. Preferably R³ is alkyl, aryl, arylalkyl, heteroalkyl, heteroaryl, or heteroarylalkyl, each unsubstituted or substituted with 1-3 substituents. The substituents are independently selected from a group that includes halo, OR, NR₂, SR, —SOR, —SO₂R, —OCOR, —NRCOR, —NRCONR₂, —NRCOOR, —OCONR₂, RCO, —COOR, —SO₃R, NRSOR, NRSO₂R, —CONR₂, SO₂NR₂, CN, CF₃, and NO₂, wherein each R is independently H or alkyl (1-4C) and with respect to any aryl or heteroaryl moiety, said group further including alkyl (1-6C) or alkenyl or alkynyl. Preferred embodiments of R³ (the substituent at position corresponding to the 2-position of the quinazoline) comprise a phenyl moiety optionally substituted with 1-2 substituents preferably halo, alkyl (1-6C), OR, NR₂, and SR wherein R is as defined above. Thus, preferred substituents at the 2-position of the quinazoline include phenyl, 2-halophenyl, e.g., 2-bromophenyl, 2-chlorophenyl, 2-fluorophenyl; 2-alkyl-phenyl, e.g., 2-methylphenyl, 2-ethylphenyl; 4-halophenyl, e.g., 4-bromophenyl, 4-chlorophenyl, 4-fluorophenyl; 5-halophenyl, e.g. 5-bromophenyl, 5-chlorophenyl, 5-fluorophenyl; 2,4- or 2,5-halophenyl, wherein the halo substituents at different positions may be identical or different, e.g. 2-fluoro-4-chlorophenyl; 2-bromo-4-chlorophenyl; 2-fluoro-5-chlorophenyl; 2-chloro-5-fluorophenyl, and the like. Other preferred embodiments of R³ comprise a cyclopentyl or cyclohexyl moiety.

As noted above, R² is a noninterfering substituent. As set forth above, a “noninterfering substituent” is one whose presence does not substantially destroy the TGF-β inhibiting ability of the compound of formula (1).

Each R² is also independently a hydrocarbyl residue (1-20C) containing 0-5 heteroatoms selected from O, S and N. Preferably, R² is independently H, alkyl, alkenyl, alkynyl, acyl or hetero-forms thereof or is aryl, arylalkyl, heteroalkyl, heteroaryl, or heteroarylalkyl, each unsubstituted or substituted with 1-3 substituents selected independently from the group consisting of alkyl, alkenyl, alkynyl, aryl, alkylaryl, aroyl, N-aryl, NH-alkylaryl, NH-aroyl, halo, OR, NR₂, SR, —SOR, —SO₂R, —OCOR, —NRCOR, —NRCONR₂, —NRCOOR, NRSOR, NRSO₂R, —OCONR₂, RCO, —COOR, —SO₃R, NRSOR, NRSO₂R, —CONR₂, SO₂NR₂, CN, CF₃, and NO₂, wherein each R is independently H or alkyl (1-4C). The aryl or aroyl groups on said substituents may be further substituted by, for example, alkyl, alkenyl, alkynyl, halo, OR, NR₂, SR, —SOR, —SO₂R, —OCOR, —NRCOR, —NRCONR₂, —NRCOOR, —OCONR₂, RCO, —COOR, —SO₃R, —CONR₂, SO₂NR₂, CN, CF₃, and NO₂, wherein each R is independently H or alkyl (1-4C). More preferably the substituents on R² are selected from R⁴, halo, OR⁴, NR₄₂, SR⁴, —OOCR⁴, —NROCR⁴, —COOR⁴, R⁴CO, —CONR⁴₂, —SO₂NR₄₂, CN, CF₃, and NO₂, wherein each R⁴ is independently H, or optionally substituted alkyl (1-6C), or optionally substituted arylalkyl (7-12C) and wherein two R⁴ or two substituents on said alkyl or arylalkyl taken together may form a fused aliphatic ring of 5-7 members.

R₂ may also, itself, be selected from the group consisting of halo, OR, NR₂, SR, —SOR, —SO₂R, —OCOR, —NRCOR, —NRCONR₂, —NRCOOR, NRSOR, NRSO₂R, —OCONR₂, RCO, —COOR, —SO₃R, NRSOR, NRSO₂R, —CONR₂, SO₂NR2, CN, CF₃, and NO₂, wherein each R is independently H or alkyl (1-4C).

More preferred substituents represented by R² are those as set forth with regard to the phenyl moieties contained in Ar′ or R³ as set forth above. Two adjacent CR² taken together may form a carbocyclic or heterocyclic fused aliphatic ring of 5-7 atoms. Preferred R² substituents are of the formula R⁴, —OR⁴, SR⁴ or R⁴NH—, especially R⁴NH—, wherein R⁴ is defined as above. Particularly preferred are instances wherein R⁴ is substituted arylalkyl. Specific representatives of the compounds of formula (1) are shown in Tables 1-3 below. All compounds listed in Table 1 have a quinazoline ring system (Z³ is N), where the A ring is unsubstituted (Z⁵-Z⁸ represent CH). The substituents of the B ring are listed in the table.

TABLE 1

| Comp und N . | L | Ar′ | R³ |
|---|---|---|---|
| 1 | NH | 4-pyridyl | 2-chlorophenyl |
| 2 | NH | 4-pyridyl | 2,6-dichlorophenyl |
| 3 | NH | 4-pyridyl | 2-methylphenyl |
| 4 | NH | 4-pyridyl | 2-bromophenyl |
| 5 | NH | 4-pyridyl | 2-fluorophenyl |
| 6 | NH | 4-pyridyl | 2,6-difluorophenyl |
| 7 | NH | 4-pyridyl | Phenyl |
| 8 | NH | 4-pyridyl | 4-fluorophenyl |
| 9 | NH | 4-pyridyl | 4-methoxyphenyl |
| 10 | NH | 4-pyridyl | 3-fluorophenyl |
| 11* | N* | 4-pyridyl | Phenyl |
| 12^{†} | N^{†} | 4-pyridyl | Phenyl |
| 13 | NHCH₂ | 4-pyridyl | Phenyl |
| 14 | NHCH₂ | 4-pyridyl | 4-chlorophenyl |
| 15 | NH | 3-pyridyl | Phenyl |
| 16 | NHCH₂ | 2-pyridyl | Phenyl |
| 17 | NHCH₂ | 3-pyridyl | Phenyl |
| 18 | NHCH₂ | 2-pyridyl | Phenyl |
| 19 | NHCH₂CH₂ | 2-pyridyl | Phenyl |
| 20 | NH | 6-pyrimidinyl | Phenyl |
| 21 | NH | 2-pyrimidinyl | Phenyl |
| 22 | NH | phenyl | Phenyl |
| 23 | NHCH₂ | phenyl | 3-chlorophenyl |
| 24 | NH | 3-hydroxyphenyl | Phenyl |
| 25 | NH | 2-hydroxyphenyl | Phenyl |
| 26 | NH | 4-hydroxyphenyl | Phenyl |
| 27 | NH | 4-indolyl | Phenyl |
| 28 | NH | 5-indolyl | Phenyl |
| 29 | NH | 4-methoxyphenyl | Phenyl |
| 30 | NH | 3-methoxyphenyl | Phenyl |
| 31 | NH | 2-methoxyphenyl | Phenyl |
| 32 | NH | 4-(2- | Phenyl |
| | | hydroxyethyl)phenyl | |
| 33 | NH | 3-cyanophenyl | Phenyl |
| 34 | NHCH₂ | 2,5-difluorophenyl | Phenyl |
| 35 | NH | 4-(2-butyl)phenyl | Phenyl |
| 36 | NHCH₂ | 4-dimethylaminophenyl | Phenyl |
| 37 | NH | 4-pyridyl | Cyclopentyl |
| 38 | NH | 2-pyridyl | Phenyl |
| 39 | NHCH₂ | 3-pyridyl | Phenyl |
| 40 | NH | 4-pyrimidyl | Phenyl |
| 41^{‡} | N^{‡} | 4-pyridyl | Phenyl |
| 42 | NH | p-aminomethylphenyl | Phenyl |
| 43 | NHCH₂ | 4-aminophenyl | Phenyl |
| 44 | NH | 4-pyridyl | 2-chlorophenyl |
| 45 | NH | phenyl | 4-pyridyl |
| 46 | NH | 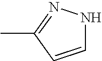 | Phenyl |
| 47 | NH | 4-pyridyl | t-butyl |
| 48 | NH | 2-benzylamino-3- | Phenyl |
| | | pyridyl | |
| 49 | NH | 2-benzylamino-4- | Phenyl |
| | | pyridyl | |
| 50 | NH | 3-benzyloxyphenyl | Phenyl |
| 51 | NH | 4-pyridyl | 3-aminophenyl |
| 52 | NH | 4-pyridyl | 4-pyridyl |
| 53 | NH | 4-pyridyl | 2-naphthyl |
| 54 | 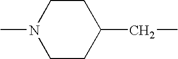 | 4-pyridyl | Phenyl |
| 55 | 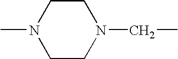 | phenyl | Phenyl |
| 56 | 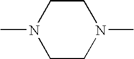 | 2-pyridyl | Phenyl |
| 57 | NHCH₂CH₂ | 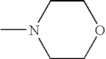 | Phenyl |
| 58 | not present | 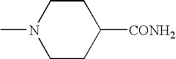 | Phenyl |
| 59 | not present | 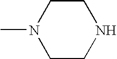 | Phenyl |
| 60 | NH | 4-pyridyl | Cyclopropyl |
| 61 | NH | 4-pyridyl | 2-trifluoromethyl |
| | | | phenyl |
| 62 | NH | 4-aminophenyl | Phenyl |
| 63 | NH | 4-pyridyl | Cyclohexyl |
| 64 | NH | 3-methoxyphenyl | 2-fluorophenyl |
| 65 | NH | 4-methoxyphenyl | 2-fluorophenyl |
| 66 | NH | 4-pyrimidinyl | 2-fluorophenyl |
| 67 | NH | 3-amino-4-pyridyl | Phenyl |
| 68 | NH | 4-pyridyl | 2- |
| | | | benzylaminophenyl |
| 69 | NH | 2-benzylaminophenyl | Phenyl |
| 70 | NH | 2-benzylaminophenyl | 4-cyanophenyl |
| 71 | NH | 3′-cyano-2- | Phenyl |
| | | benzylaminophenyl | |

*R¹ = 2-propyl
^{†}R¹ = 4-methoxyphenyl
^{‡}R¹ = 4-methoxybenzyl

The compounds in Table 2 contain modifications of the quinazoline nucleus as shown. All of the compounds in Table 2 are embodiments of formula (1) wherein Z³ is N and Z⁶ and Z⁷ represent CH. In all cases the linker, L, is present and is NH.

TABLE 2

| Compound | | | | |
|---|---|---|---|---|
| No. | Z⁵ | Z⁸ | Ar′ | R³ |
| 72 | CH | N | 4-pyridyl | 2-fluorophenyl |
| 73 | CH | N | 4-pyridyl | 2-chlorophenyl |
| 74 | CH | N | 4-pyridyl | 5-chloro-2- |
| | | | | fluorphenyl |
| 75 | CH | N | 4-(3-methyl)- | 5-chloro-2- |
| | | | pyridyl | fluorphenyl |
| 76 | CH | N | 4-pyridyl | Phenyl |
| 77 | N | N | 4-pyridyl | phenyl |
| 78 | N | CH | 4-pyridyl | Phenyl |
| 79 | N | N | 4-pyridyl | 5-chloro-2- |
| | | | | fluorphenyl |
| 80 | N | N | 4-(3-methyl)- | 5-chloro-2- |
| | | | pyridyl | fluorphenyl |
| 81 | N | N | 4-pyridyl | 2-chlorophenyl |

Additional compounds were prepared wherein ring A contains CR2 at Z6 or Z⁷ where R² is not H. These compounds, which are all quinazoline derivatives, wherein L is NH and Ar′ is 4-pyridyl, are shown in Table 3.

TABLE 3

| Compound | | |
|---|---|---|
| No. | R³ | CR² as noted |
| 82 | 2-chlorophenyl | 6,7-dimethoxy |
| 83 | 2-fluorophenyl | 6-nitro |
| 84 | 2-fluorophenyl | 6-amino |
| 85 | 2-fluorophenyl | 7-amino |
| 86 | 2-fluorophenyl | 6-(3-methoxybenzylamino) |
| 87 | 2-fluorophenyl | 6-(4-methoxybenzylamino) |
| 88 | 2-fluorophenyl | 6-(2-isobutylamino) |
| 89 | 2-fluorophenyl | 6-(4- |
| | | methylmercaptobenzylamino) |
| 90 | 2-fluorophenyl | 6-(4-methoxybenzoyl amino) |
| 91 | 4-fluorophenyl | 7-amino |
| 92 | 4-fluorophenyl | 7-(3-methoxybenzylamino) |

Although the invention is illustrated with reference to certain quinazoline derivatives, it is not so limited. Inhibitors of the present invention include compounds having a non-quinazoline, such as, a pyridine, pyrimidine nucleus carrying substituents like those discussed above with respect to the quinazoline derivatives.

The compounds of the invention, including compounds of the formula (1) may be supplied in the form of their pharmaceutically acceptable acid-addition salts including salts of inorganic acids such as hydrochloric, sulfuric, hydrobromic, or phosphoric acid or salts of organic acids such as acetic, tartaric, succinic, benzoic, salicylic, and the like. If a carboxyl moiety is present on the compound of formula (1), the compound may also be supplied as a salt with a pharmaceutically acceptable cation.

Another group of compounds for use in the methods of the present invention is represented by the following formula (2)

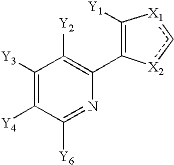

wherein Y1 is phenyl or naphthyl optionally substituted with one or more substituents selected from halo, alkoxy(1-6 C), alkylthio(1-6 C), alkyl(1-6 C), haloalkyl (1-6C), —O—(CH2)m-Ph, —S—(CH2)m-Ph, cyano, phenyl, and CO2R, wherein R is hydrogen or alkyl(1-6 C), and m is 0-3; or phenyl fused with a 5- or 7-membered aromatic or non-aromatic ring wherein said ring contains up to three heteroatoms, independently selected from N, O, and S:

Y2, Y3, Y4, and Y5 independently represent hydrogen, alkyl(1-6C), alkoxy(1-6 C), haloalkyl(1-6 C), halo, NH₂, NH-alkyl(1-6C), or NH(CH₂)ₙ-Ph wherein n is 0-3; or an adjacent pair of Y₂, Y₃, Y₄, and Y₅ form a fused 6-membered aromatic ring optionally containing up to 2 nitrogen atoms, said ring being optionally substituted by one o more substituents independently selected from alkyl(1-6 C), alkoxy(a-6 C), haloalkyl(1-6 C), halo, NH₂, NH-alkyl(1-6 C), or NH(CH₂)ₙ-Ph, wherein n is 0-3, and the remainder of Y₂, Y₃, Y₄, and Y₅ represent hydrogen, alkyl(1-6 C), alkoxy(1-6C), haloalkyl(1-6 C), halo, NH₂, NH-alkyl(1-6 C), or NH(CH₂)ₙ-Ph wherein n is 0-3; and

one of X₁ and X₂ is N and the other is NR₆, wherein R₆ is hydrogen or alkyl(1-6 C).

As used in formula (2), the double bonds indicated by the dotted lined represent possible tautomeric ring forms of the compounds. Further information about compounds of formula (2) and their preparation is disclosed in WO 02/40468, published May 23, 2002, the entire disclosure of which is hereby expressly incorporated by reference.

Yet another group of compounds for use in the methods of the invention is represented by the following formula (3)

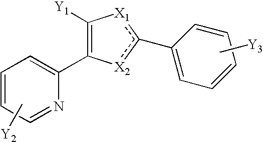

wherein Y1 is naphthyl, anthracenyl, or phenyl optionally substituted with one or more substituents selected from the group consisting of halo, alkoxy(1-6 C), alkylthio(1-6 C), alkyl(1-6 C), —O—(CH2)-Ph, —S—(CH2)n-Ph, cyano, phenyl, and CO2R,

wherein R is hydrogen or alkyl(1-6 C), and n is 0, 1, 2, or 3; or Y1 represents phenyl fused with an aromatic or non-aromatic cyclic ring of 5-7 members wherein said cyclic ring optionally contains up to two heteroatoms, independently selected from N, O, and S;

Y2 is H, NH(CH2)n-Ph or NH-alkyl(1-6 C), wherein n is 0, 1, 2, or 3;

Y3 is CO2H, CONH2, CN, N02, alkylthio(1-6 C), —SO₂-alkyl(C1-6), alkoxy(C1-6), SONH2, CONHOH, NH₂, CHO, CH2NH2, or CO2R, wherein R is hydrogen or alkyl(1-6 C);

one of X1 and X2 is N or CR′, and other is NR′ or CHR′ wherein R′ is hydrogen, OH, alkyl(C-16), or cycloalkyl(C3-7); or when one of X1 and X2 is N or CR′ then the other may be S or O.

Further details of the compounds of formula (3) and their modes of preparation are disclosed in WO 00/61576 published Oct. 19, 2000, the entire disclosure of which is hereby expressly incorporated by reference.

In a further embodiment, the TGF-β inhibitors of the present invention are represented by the following formula (4)

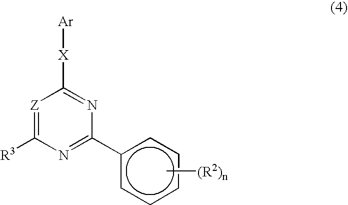

and the pharmaceutically acceptable salts and prodrug forms thereof; wherein

Ar represents an optionally substituted aromatic or optionally substituted heteroaromatic moiety containing 5-12 ring members wherein said heteroaromatic moiety contains one or more O, S, and/or N with a proviso that the optionally substituted Ar is not

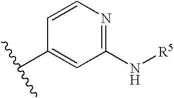

wherein R5 is H, alkyl (1-6C), alkenyl (2-6C), alkynyl (2-6C), an aromatic or heteroaromatic moiety containing 5-11 ring members;

X is NR¹, O, or S;

R¹ is H, alkyl (1-8C), alkenyl (2-8C), or alkynyl (2-8C);

Z represents N or CR⁴;

each of R³ and R⁴ is independently H, or a non-interfering substituent;

each R² is independently a non-interfering substituent; and

n is 0, 1, 2, 3, 4, or 5. In one embodiment, if n>2, and the R²'s are adjacent, they can be joined together to form a 5 to 7 membered non-aromatic, heteroaromatic, or aromatic ring containing 1 to 3 heteroatoms where each heteroatom can independently be O, N, or S.

In preferred embodiments, Ar represents an optionally substituted aromatic or optionally substituted heteroaromatic moiety containing 5-9 ring members wherein said heteroaromatic moiety contains one or more N; or

R1 is H, alkyl (1-8C), alkenyl (2-8C), or alkynyl (2-8C); or

Z represents N or CR4; wherein

R⁴ is H, alkyl (1-10C), alkenyl (2-10C), or alkynyl (2-10C), acyl (1-10C), aryl, alkylaryl, aroyl, O-aryl, O-alkylaryl, O-aroyl, NR-aryl, NR-alkylaryl, NR-aroyl, or the hetero forms of any of the foregoing, halo, OR, NR₂, SR, —SOR, —NRSOR, —NRSO₂R, —SO₂R, —OCOR, —NRCOR, —NRCONR₂, —NRCOOR, —OCONR₂, —COOR, —SO₃R, —CONR₂, —SO₂NR₂, —CN, —CF₃, or —NO₂, wherein each R is independently H or alkyl (1-10C) or a halo or heteroatom-containing form of said alkyl, each of which may optionally be substituted. Preferably R⁴ is H, alkyl (1-10C), OR, SR or NR₂ wherein R is H or alkyl (1-10C) or is O-aryl; or

R³ is defined in the same manner as R⁴ and preferred forms are similar, but R³ is independently embodied; or

each R² is independently alkyl (1-8C), alkenyl (2-8C), alkynyl (2-8C), acyl (1-8C), aryl, alkylaryl, aroyl, O-aryl, O-alkylaryl, O-aroyl, NR-aryl, NR-alkylaryl, NR-aroyl, or the hetero forms of any of the foregoing, halo, OR, NR₂, SR, —SOR, —NRSOR, —NRSO₂R, —NRSO₂R₂, —SO₂R, —OCOR, —OSO₃R, —NRCOR, —NRCONR₂, —NRCOOR, —OCONR₂, —COOR, —SO₃R, —CONR₂, SO₂NR₂, —CN, —CF₃, or —NO₂, wherein each R is independently H or lower alkyl (1-4C). Preferably R² is halo, alkyl (1-6C), OR, SR or NR₂ wherein R is H or lower alkyl (1-4C), more preferably halo; or n is 0-3.

The optional substituents on the aromatic or heteroaromatic moiety represented by Ar include alkyl (1-10C), alkenyl (2-10C), alkynyl (2-10C), acyl (1-10C), aryl, alkylaryl, aroyl, O-aryl, O-alkylaryl, O-aroyl, NR-aryl, NR-alkylaryl, NR-aroyl, or the hetero forms of any of the foregoing, halo, OR, NR2, SR, —SOR, —NRSOR, —NRSO₂R, —SO₂R, —OCOR, —NRCOR, —NRCONR₂, —NRCOOR, —OCONR₂, —COOR, —SO₃R, —CONR₂, —SO₂NR₂, —CN, —CF₃, and/or NO₂, wherein each R is independently H or lower alkyl (1-4C). Preferred substituents include alkyl, OR, NR₂, O-alkylaryl and NH-alkylaryl.

In general, any alkyl, alkenyl, alkynyl, acyl, or aryl group contained in a substituent may itself optionally be substituted by additional substituents. The nature of these substituents is similar to those recited with regard to the primary substituents themselves.

Representative compounds of formula (4) are listed in the following Table 4.

| COM- | |
|---|---|
| POUND # | STRUCTURE |
| 93 | 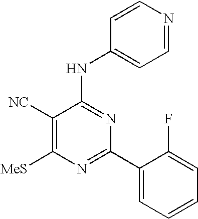 |
| 94 | 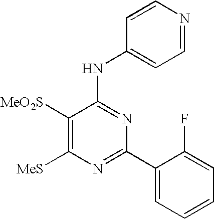 |
| 95 | 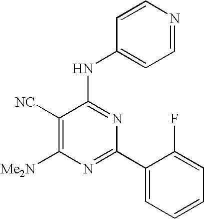 |
| 96 | 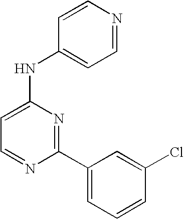 |
| 97 | 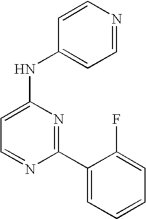 |
| 98 | 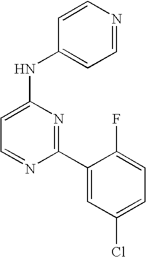 |
| 99 | 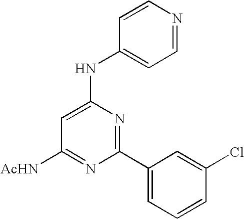 |
| 100 | 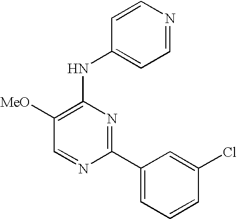 |
| 101 | 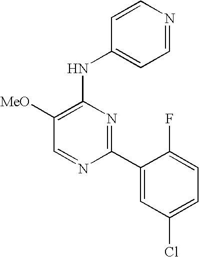 |
| 102 | 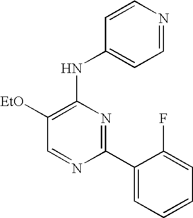 |
| 103 | 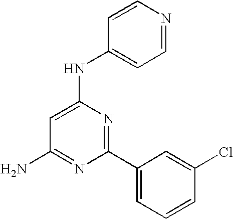 |
| 104 | 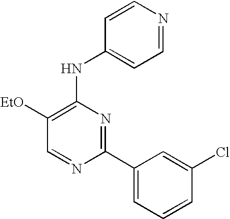 |
| 105 | 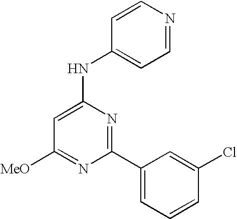 |
| 106 | 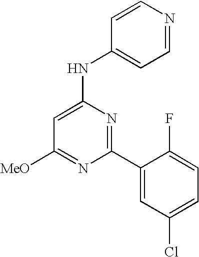 |
| 107 | 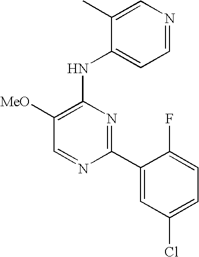 |
| 108 | 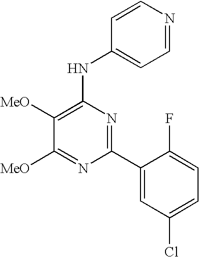 |
| 109 | 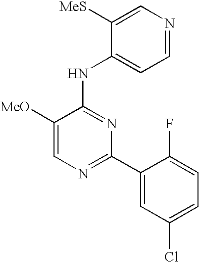 |
| 110 | 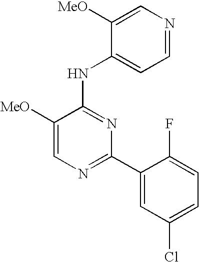 |
| 111 | 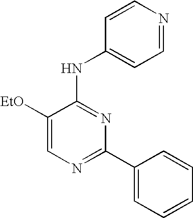 |
| 112 | 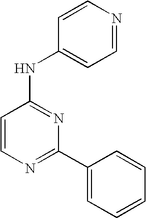 |
| 113 | 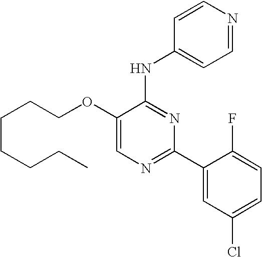 |
| 114 | 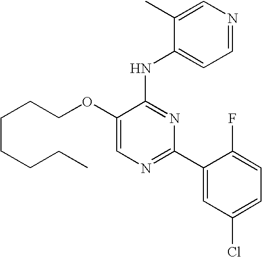 |
| 115 | 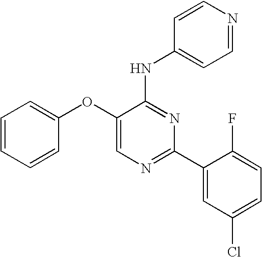 |
| 116 | 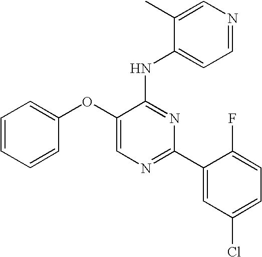 |
| 117 | 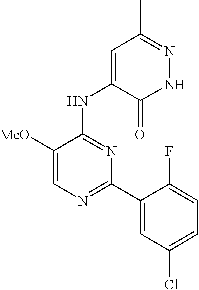 |
| 118 | 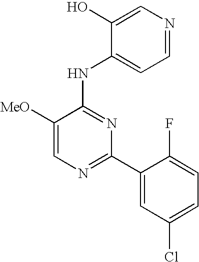 |
| 119 | 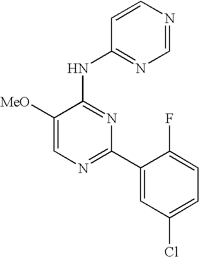 |
| 120 | 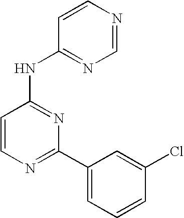 |
| 121 | 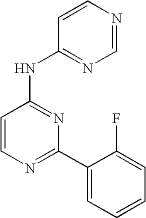 |
| 122 | 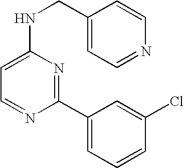 |
| 123 | 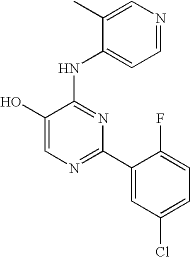 |
| 124 | 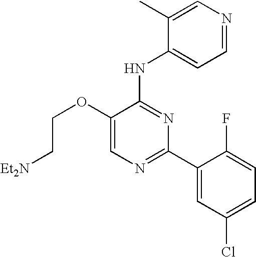 |
| 125 | 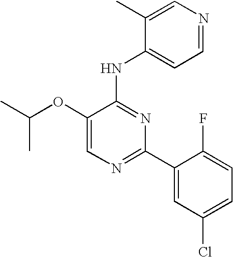 |
| 126 | 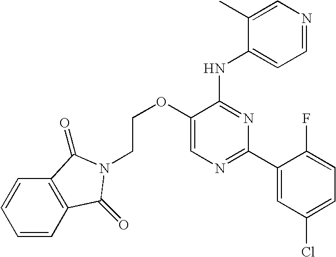 |
| 127 | 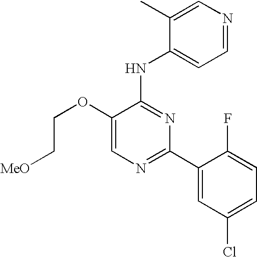 |
| 128 | 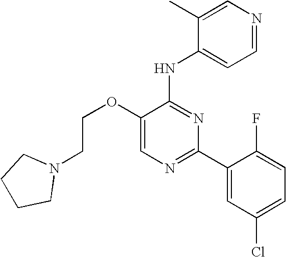 |
| 129 | 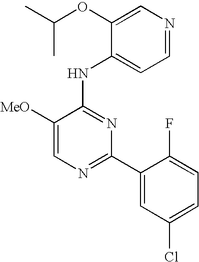 |
| 130 | 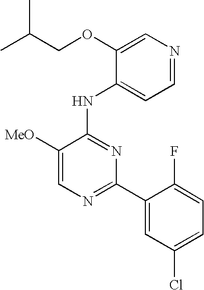 |
| 131 | 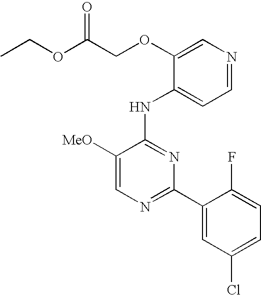 |
| 132 | 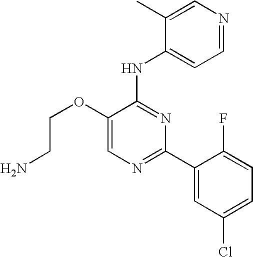 |
| 133 | 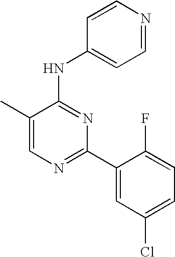 |
| 134 | 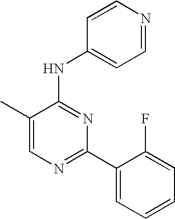 |
| 135 | 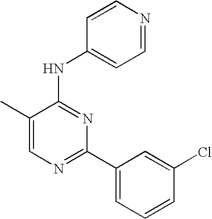 |
| 136 | 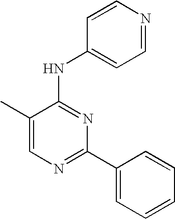 |
| 137 | 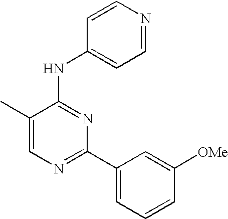 |
| 138 | 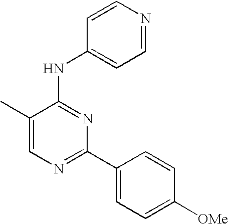 |
| 139 | 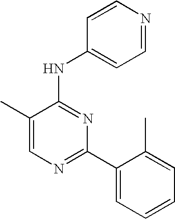 |
| 140 | 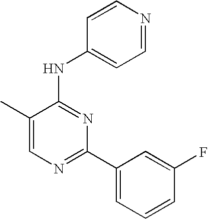 |
| 141 | 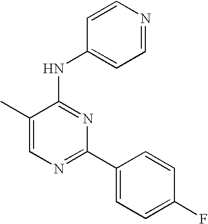 |
| 142 | 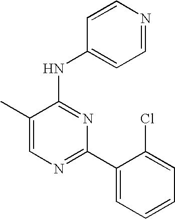 |
| 143 | 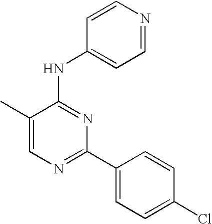 |
| 144 | 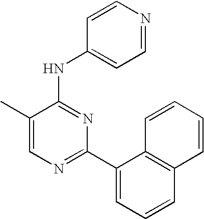 |
| 145 | 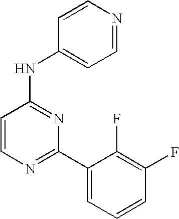 |
| 146 | 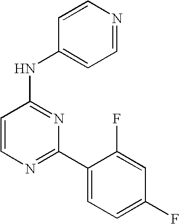 |
| 147 | 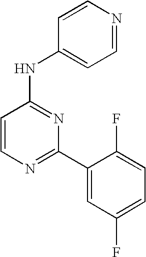 |
| 148 | 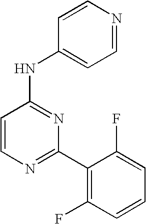 |
| 149 | 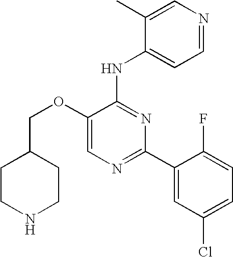 |
| 150 | 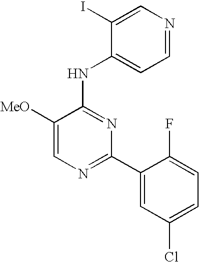 |
| 151 | 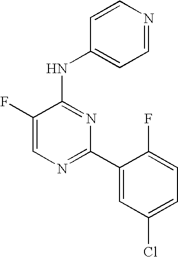 |
| 152 | 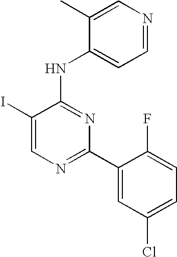 |
| 153 | 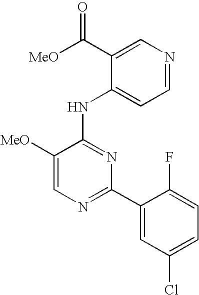 |
| 154 | 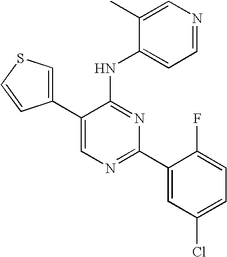 |
| 155 | 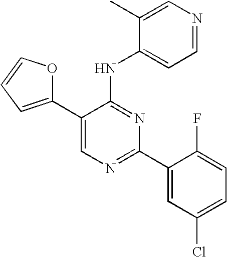 |
| 156 | 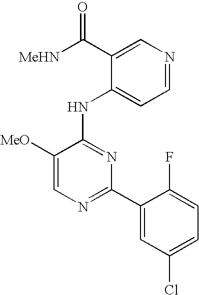 |
| 157 | 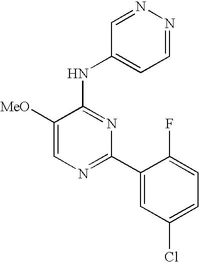 |
| 158 | 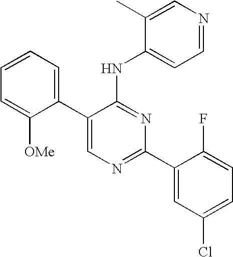 |
| 159 | 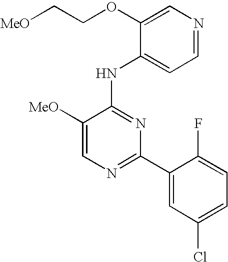 |
| 160 | 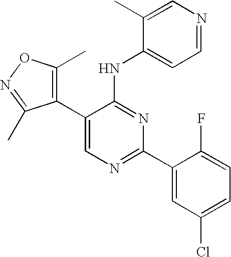 |
| 161 | 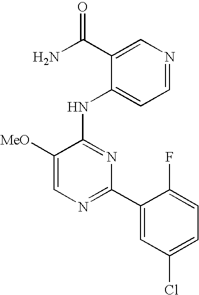 |
| 162 | 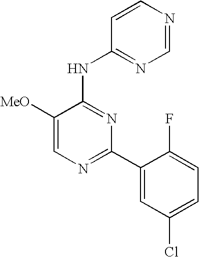 |
| 163 | 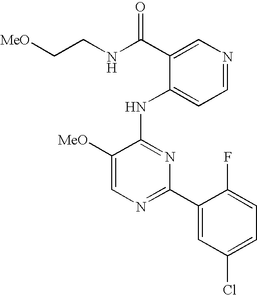 |
| 164 | 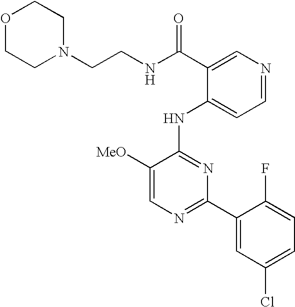 |
| 165 | 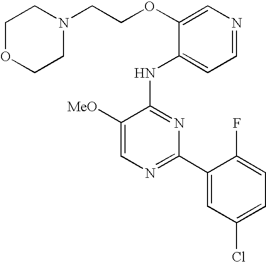 |
| 166 | 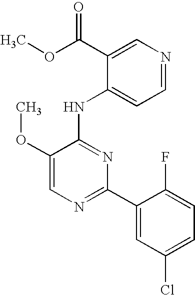 |
| 167 | 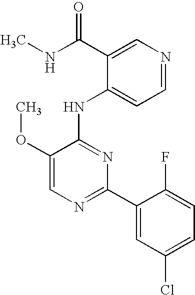 |
| 168 | 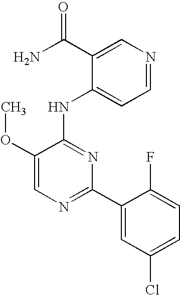 |
| 169 | 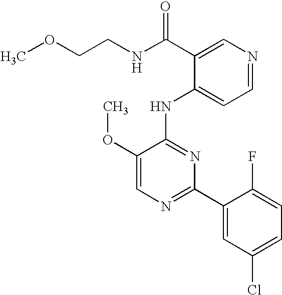 |
| 170 | 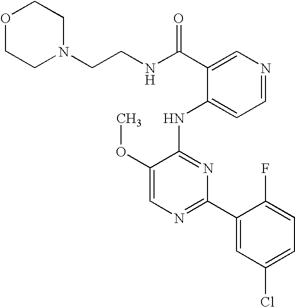 |
| 171 | 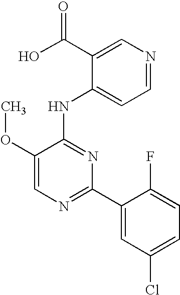 |
| 172 | 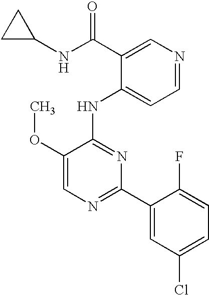 |
| 173 | 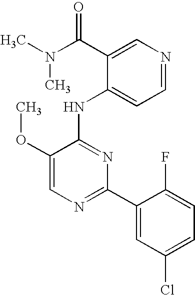 |
| 174 | 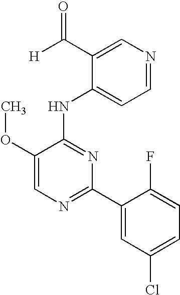 |
| 175 | 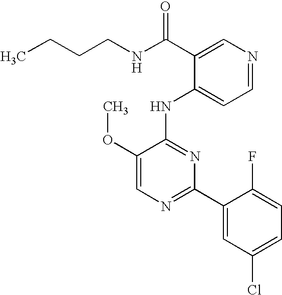 |
| 176 | 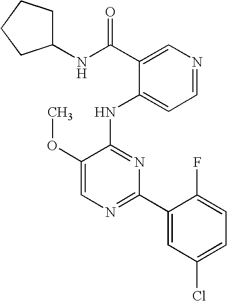 |
| 177 | 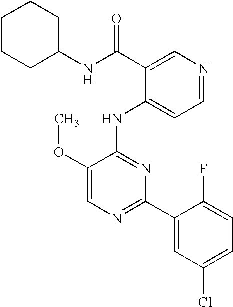 |
| 178 | 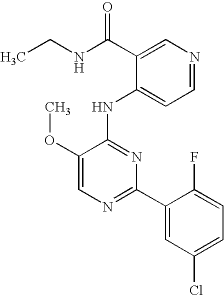 |
| 179 | 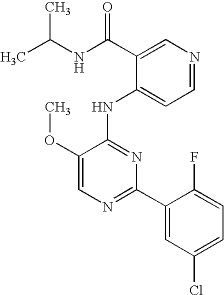 |
| 180 | 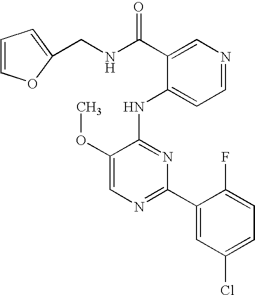 |
| 181 | 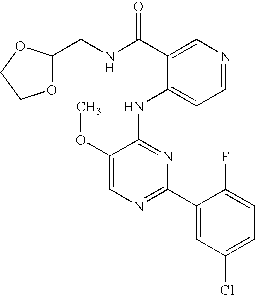 |
| 182 | 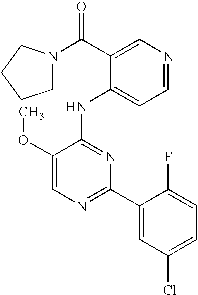 |
| 183 | 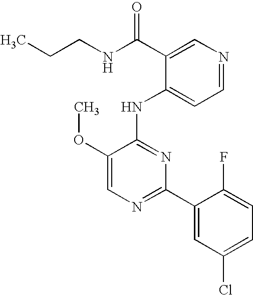 |
| 184 | 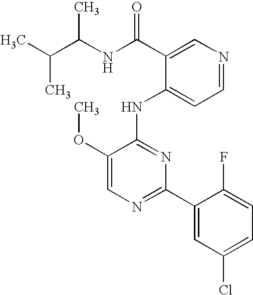 |
| 185 | 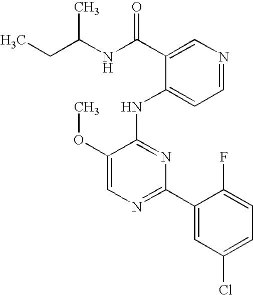 |
| 186 | 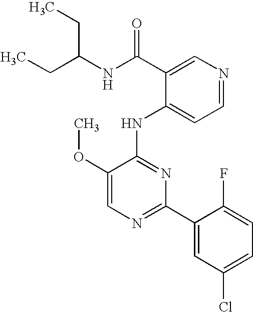 |
| 187 | 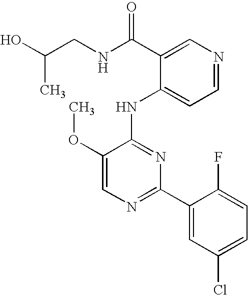 |
| 188 | 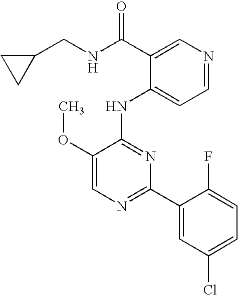 |
| 189 | 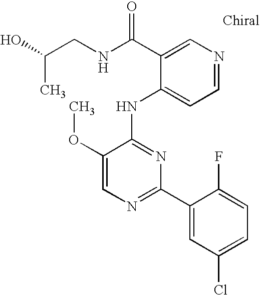 |
| 190 | 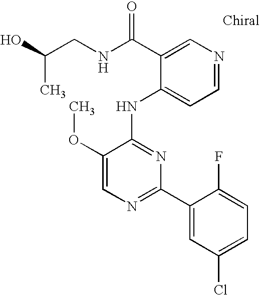 |
| 191 | 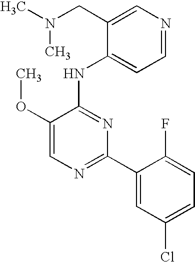 |
| 192 | 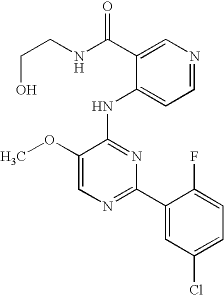 |
| 193 | 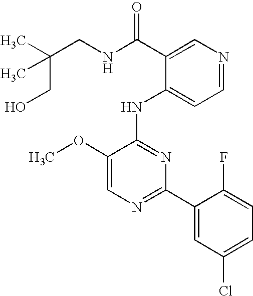 |
| 194 | 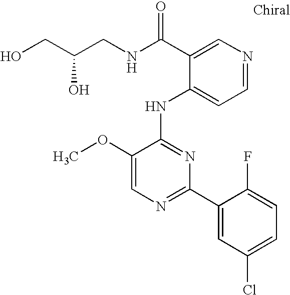 |
| 195 | 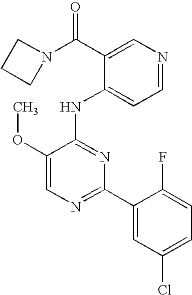 |
| 196 | 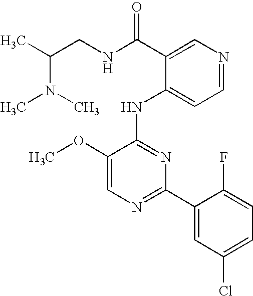 |
| 197 | 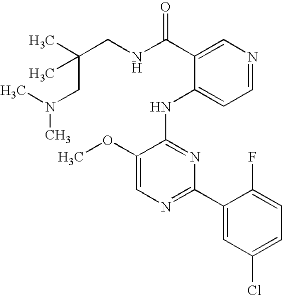 |
| 198 | 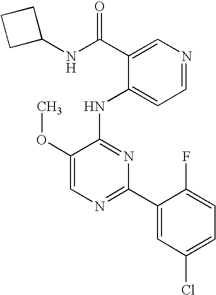 |
| 199 | 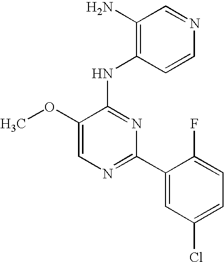 |
| 200 | 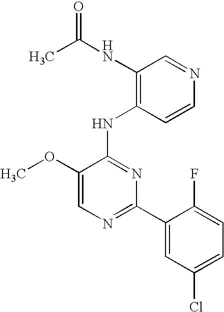 |
| 201 | 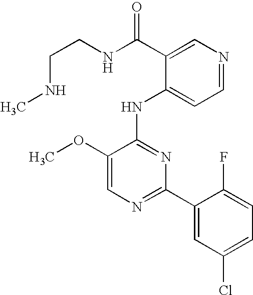 |
| 202 | 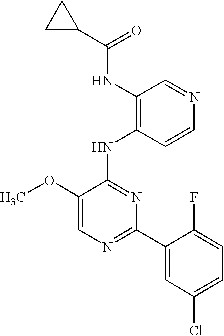 |
| 203 | 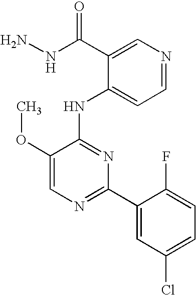 |
| 204 | 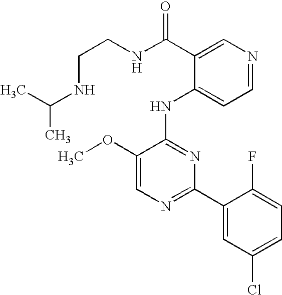 |
| 205 | 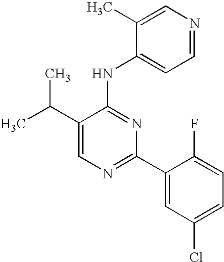 |
| 206 | 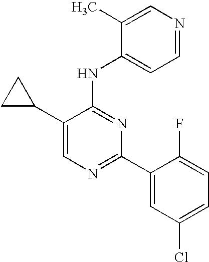 |
| 207 | 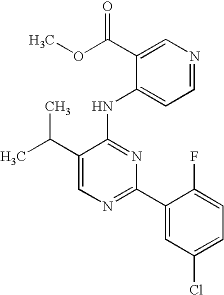 |
| 208 | 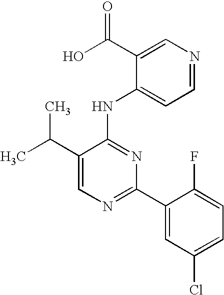 |
| 209 | 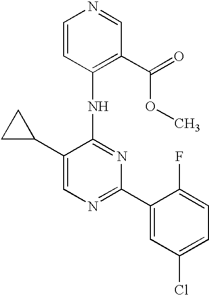 |
| 210 | 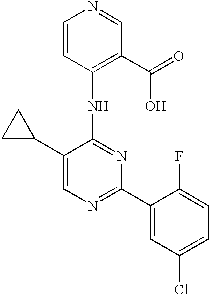 |
| 211 | 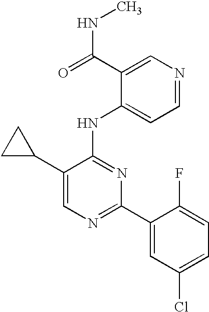 |
| 212 | 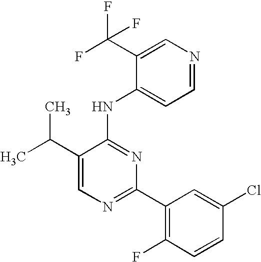 |
| 213 | 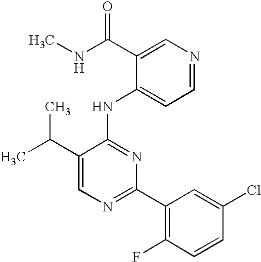 |
| 214 | 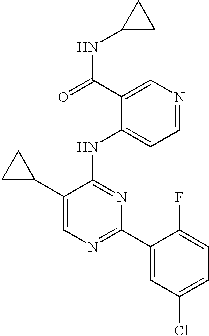 |
| 215 | 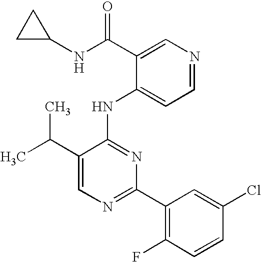 |
| 216 | 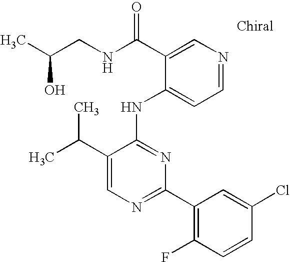 |
| 217 | 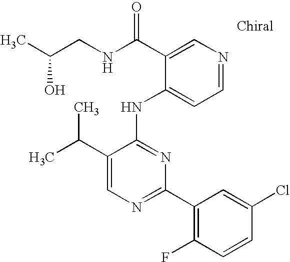 |
| 218 | 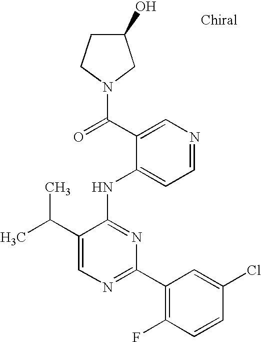 |
| 219 | 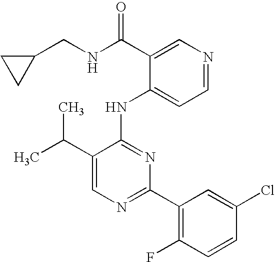 |
| 220 | 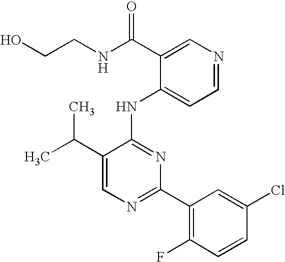 |
| 221 | 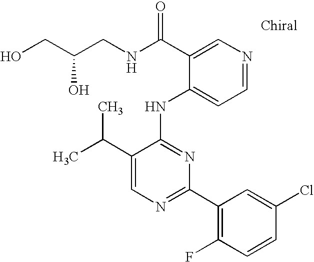 |
| 222 | 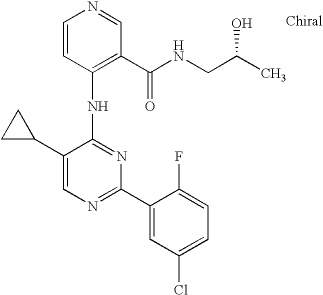 |
| 223 | 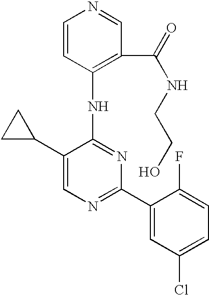 |
| 224 | 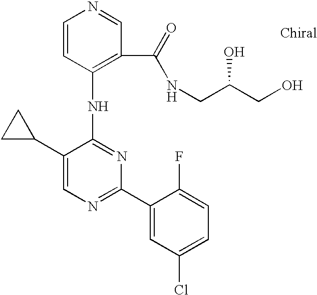 |
| 225 | 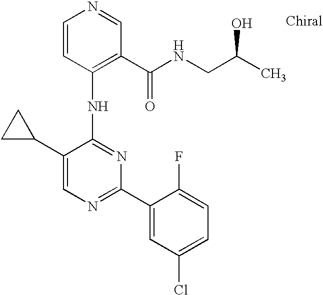 |
| 226 | 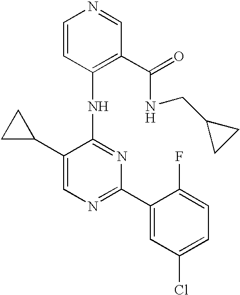 |
| 227 | 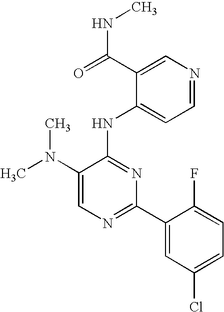 |
| 228 | 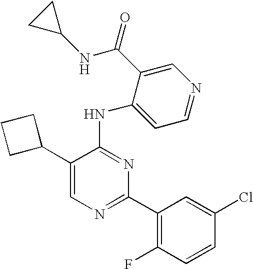 |
| 229 | 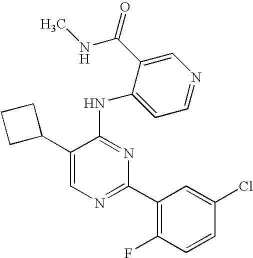 |
| 230 | 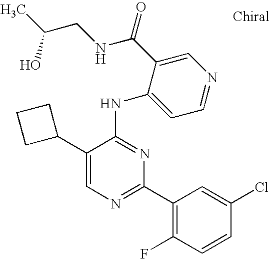 |
| 231 | 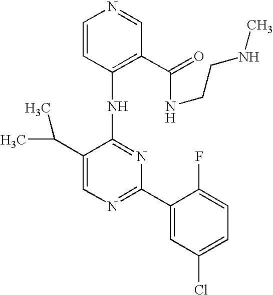 |
| 232 | 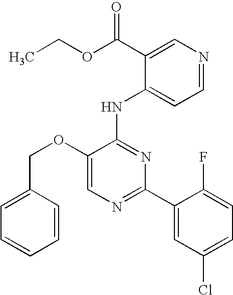 |
| 233 | 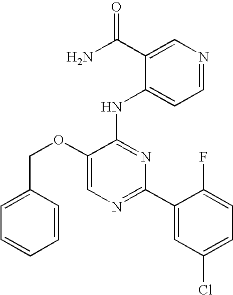 |
| 234 | 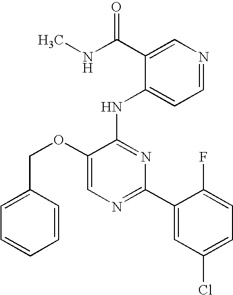 |
| 235 | 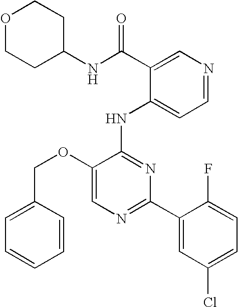 |
| 236 | 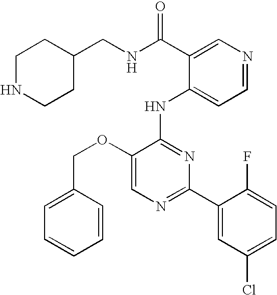 |
| 237 | 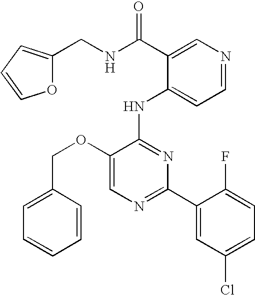 |
| 238 | 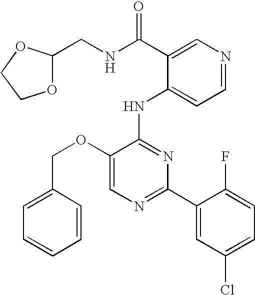 |
| 239 | 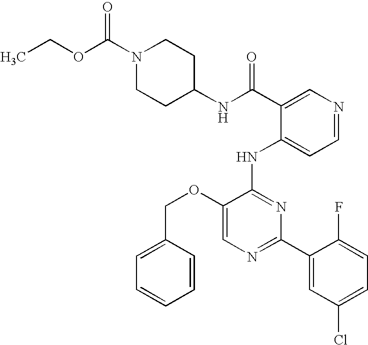 |
| 240 | 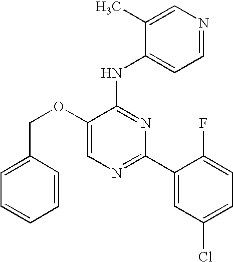 |
| 241 | 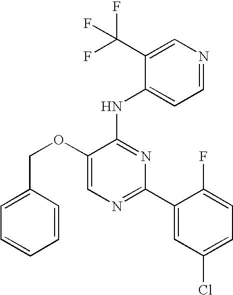 |
| 242 | 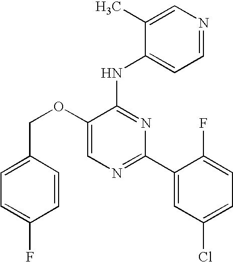 |
| 243 | 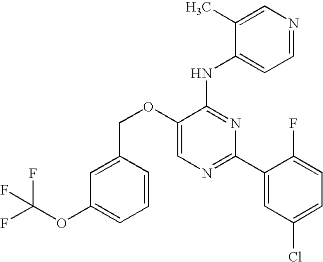 |
| 244 | 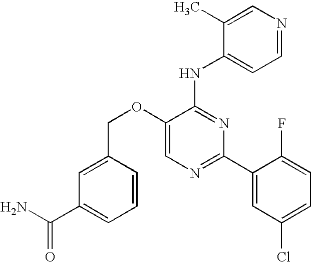 |
| 245 | 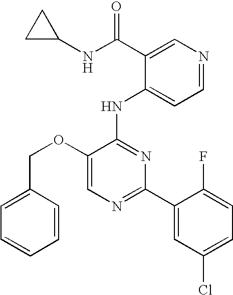 |
| 246 | 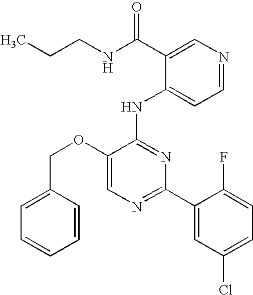 |
| 247 | 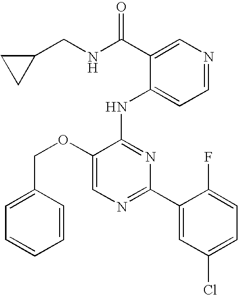 |
| 248 | 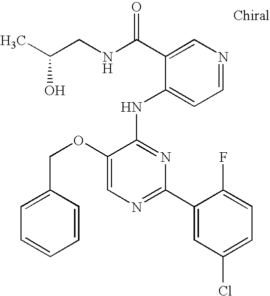 |
| 249 | 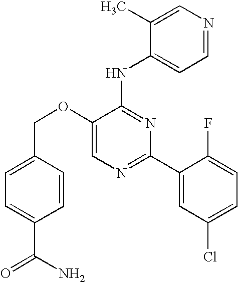 |
| 250 | 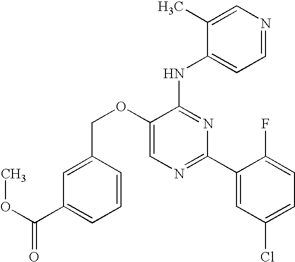 |
| 251 | 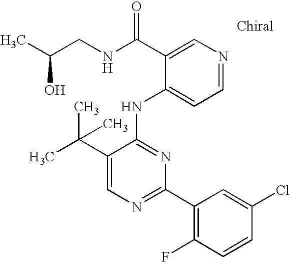 |
| 252 | 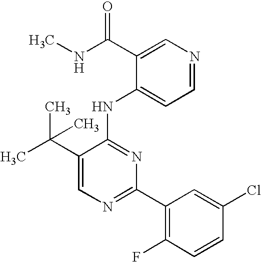 |

Further TGF-β inhibitors for use in the methods of the present invention are represented by formula (5)

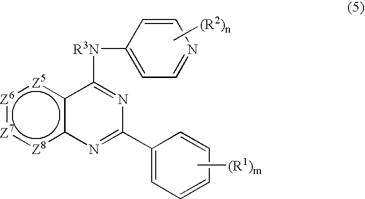

or pharmaceutically acceptable salts thereof;

wherein each of Z⁵, Z⁶, Z⁷ and Z⁸ is N or CH and wherein one or two Z⁵, Z⁶, Z⁷ and Z⁸ are N and wherein two adjacent Z positions cannot be N;

wherein m and n are each independently 0-3;

wherein two adjacent R′ groups may be joined to form an aliphatic heterocyclic ring of 5-6 members;

wherein R² is a noninterfering substituent; and

wherein R³ is H or CH₃.

Representative compound of formula (5) are listed in the following Table 5.

TABLE 5

| COM- | |
|---|---|
| POUND # | STRUCTURE |
| 253 | 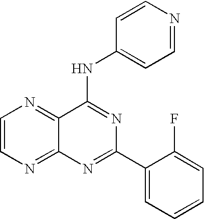 |
| 254 | 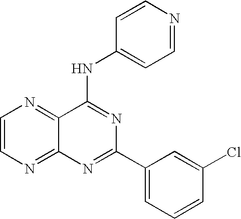 |
| 255 | 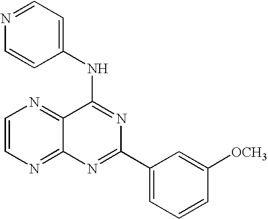 |
| 256 | 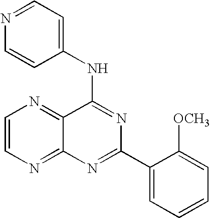 |
| 257 | 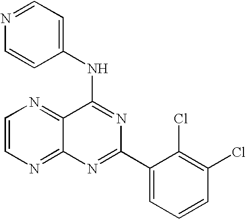 |
| 258 | 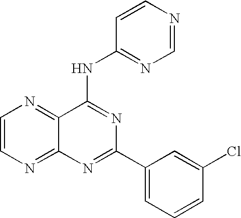 |
| 259 | 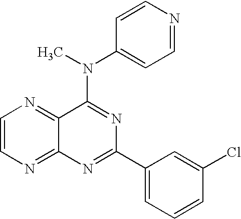 |
| 260 | 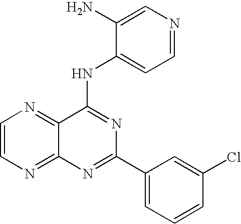 |
| 261 | 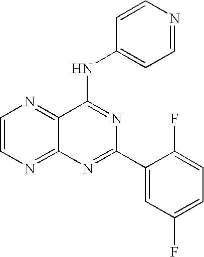 |
| 262 | 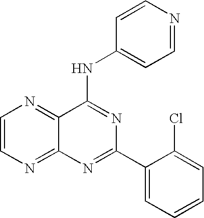 |
| 263 | 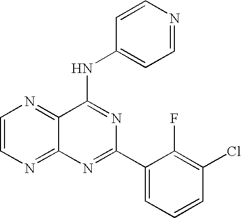 |
| 264 | 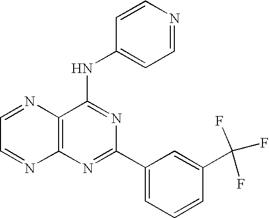 |
| 265 | 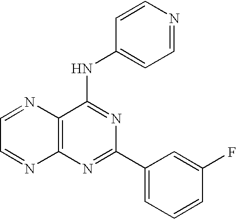 |
| 266 | 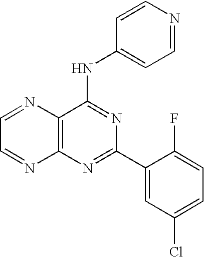 |
| 267 | 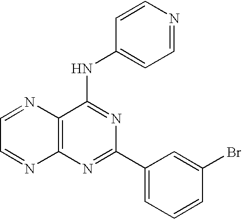 |
| 268 | 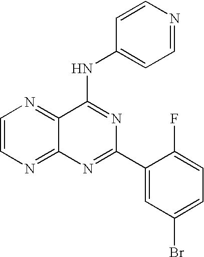 |
| 269 | 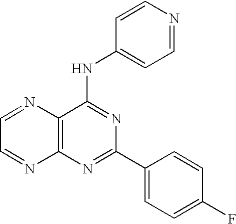 |
| 270 | 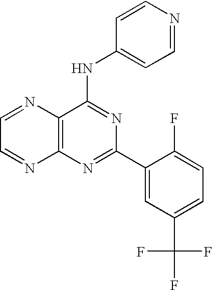 |
| 271 | 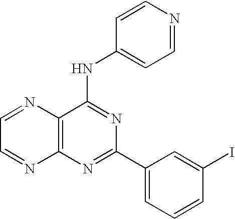 |
| 272 | 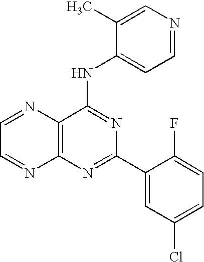 |
| 273 | 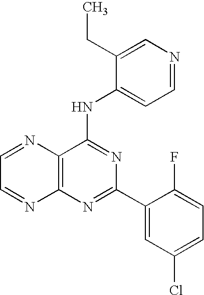 |
| 274 | 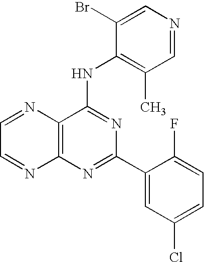 |
| 275 | 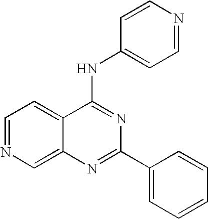 |
| 276 | 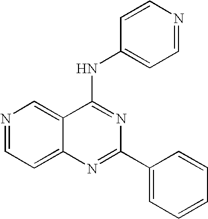 |
| 277 | 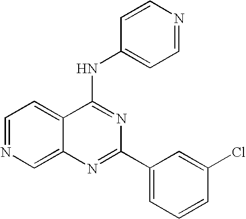 |
| 278 | 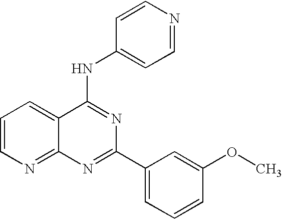 |
| 279 | 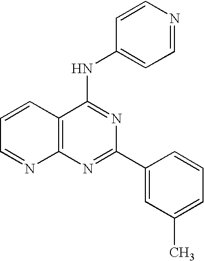 |
| 280 | 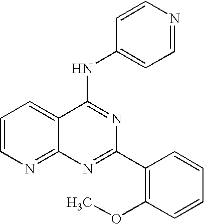 |
| 281 | 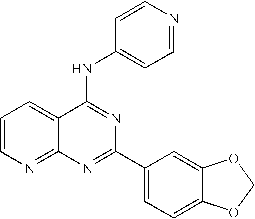 |
| 282 | 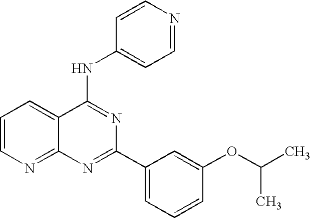 |
| 283 | 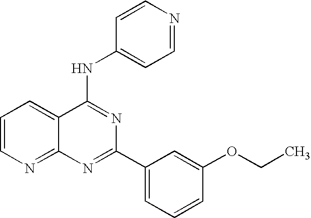 |
| 284 | 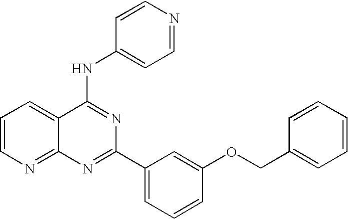 |
| 285 | 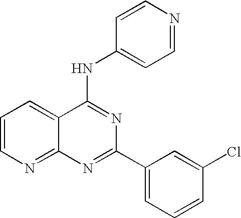 |
| 286 | 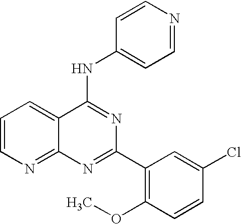 |
| 287 | 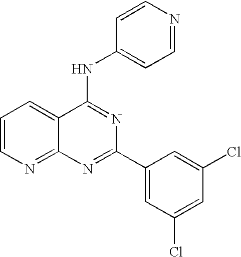 |
| 288 | 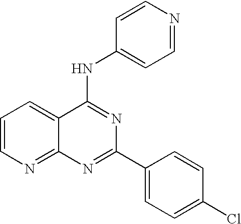 |
| 289 | 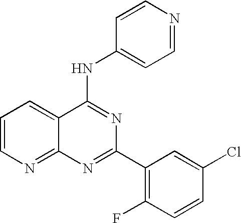 |
| 290 | 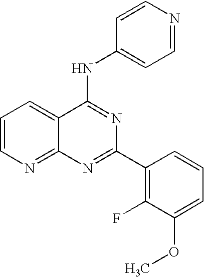 |
| 291 | 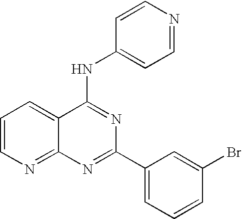 |
| 292 | 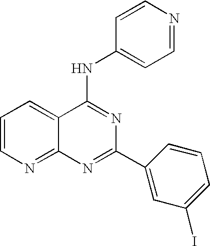 |
| 293 | 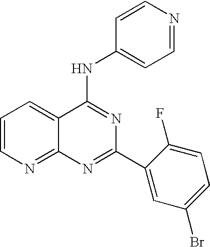 |
| 294 | 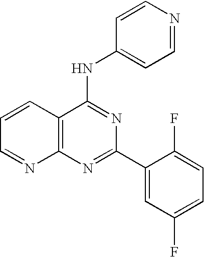 |
| 295 | 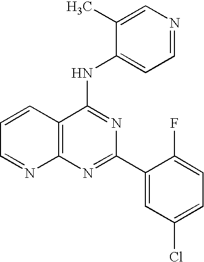 |
| 296 | 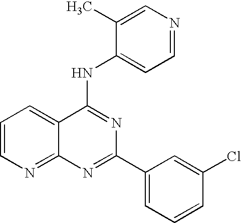 |

The TGF-β inhibitors herein can also be supplied in the form of a “prodrug” which is designed to release the compounds when administered to a subject. Prodrug form designs are well known in the art, and depend on the substituents contained in the compound. For example, a substituent containing sulfhydryl could be coupled to a carrier which renders the compound biologically inactive until removed by endogenous enzymes or, for example, by enzymes targeted to a particular receptor or location in the subject.

In the event that any of the substituents of the foregoing compounds contain chiral centers, as some, indeed, do, the compounds include all stereoisomeric forms thereof, both as isolated stereoisomers and mixtures of these stereoisomeric forms.

Synthesis of Compounds of the Invention

The small molecule compounds of formula (1) of the invention may be synthesized from the corresponding 4-halo-2-phenyl quinazoline as described in Reaction Scheme 1; which may be obtained from the corresponding 4-hydroxyquinazoline as shown in Reaction Scheme 2. Alternatively, the compounds can be prepared using anthranylamide as a starting material and benzoylating the amino group followed by cyclization to obtain the intermediate 2-phenyl-4-hydroxy quinazoline as shown in Reaction Scheme 3. Reaction Schemes 4-6 are similar to Reaction Scheme 3 except that an appropriate pyridine or 1,4-pyrimidine nucleus, substituted with a carboxamide residue and an adjacent amino residue, is substituted for the anthranylimide. The compounds of the invention wherein R1 is H can be further derivatized to comprise other embodiments of R¹ as shown in Reaction Scheme 7.

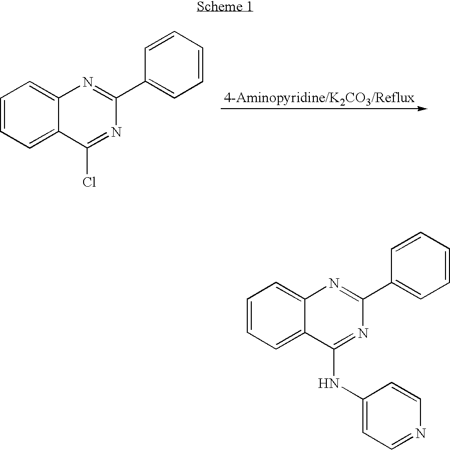

Reaction Scheme 1 is illustrative of the simple conversion of a halogenated quinazoline to compounds of the invention. Of course, the phenyl of the illustration at position 2 may be generalized as R³ and the 4-pyridylamino at position 2 can be generalized to Ar′-L or Ar′-.

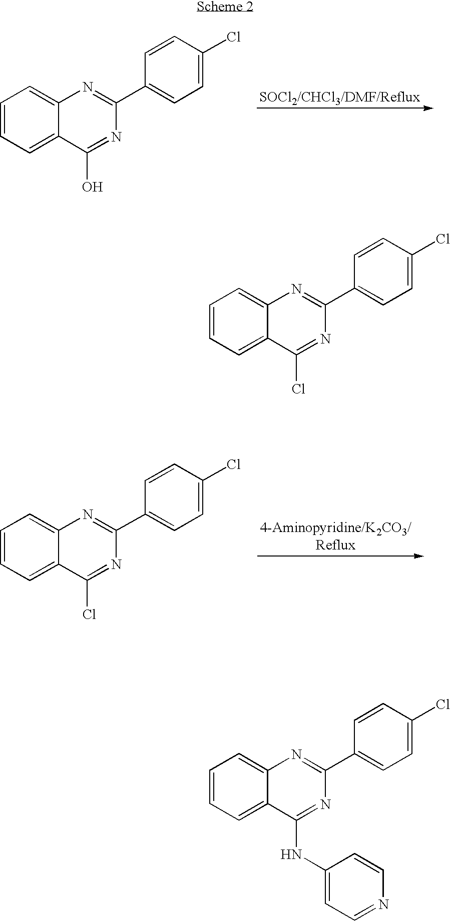

Reaction Scheme 2 can, of course, be generalized in the same manner as set forth for Reaction Scheme 1.

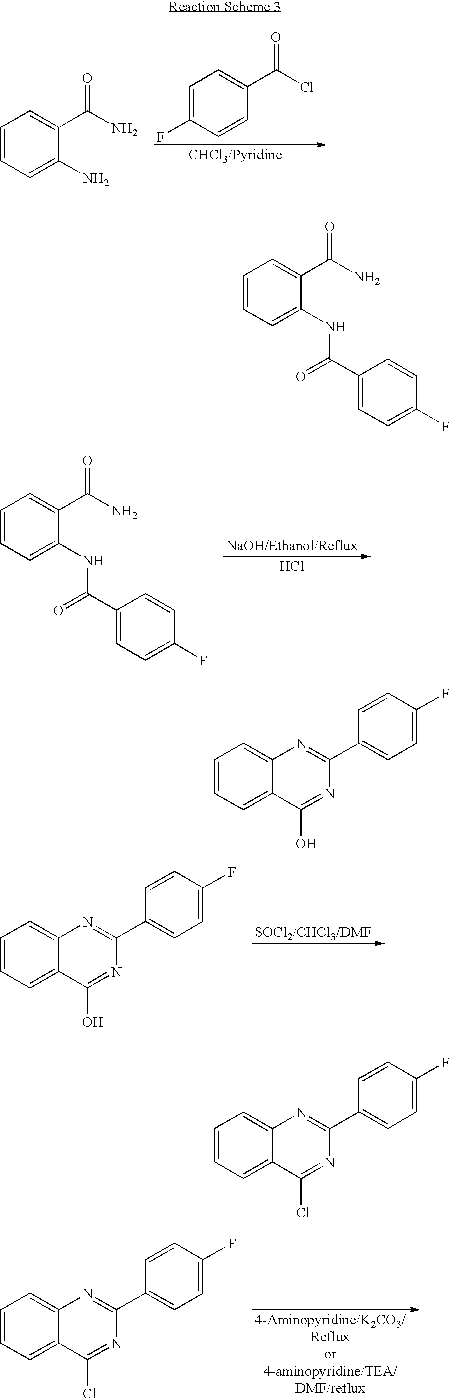

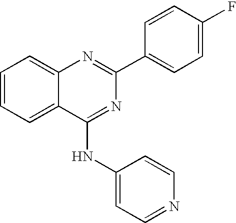

Again, Reaction Scheme 3 can be generalized by substituting the corresponding acyl halide, R³COCl for the parafluorobenzoyl chloride. Further, Ar′ or Ar′-L may be substituted for 4-aminopyridine in the last step.

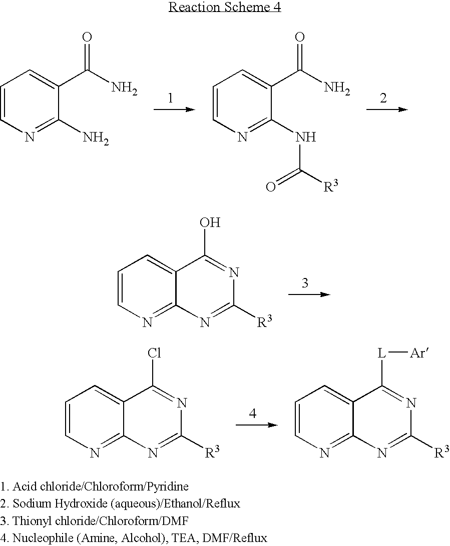

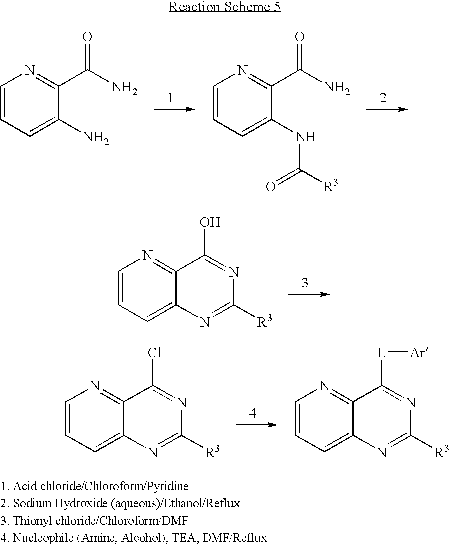

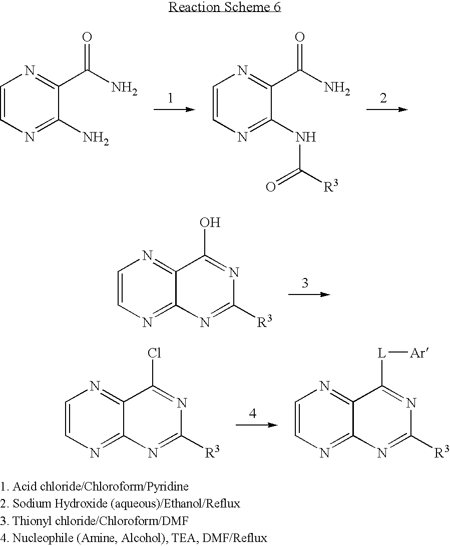

It is seen that Reaction Scheme 1 represents the last step of Reaction Schemes 2-6 and that Reaction Scheme 2 represents the last two steps of Reaction Scheme 3-6.

Reaction Scheme 7 provides conditions wherein compounds of formula (1) are obtained wherein R¹ is other than H.

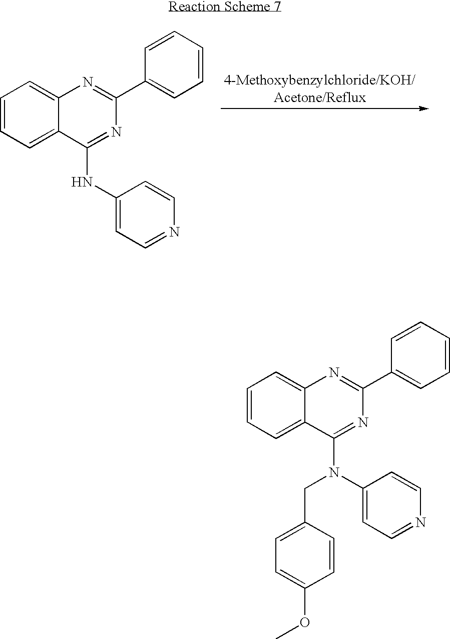

Reaction Scheme 8 is a modification of Reaction Scheme 3 which simply demonstrates that substituents on ring A are carried through the synthesis process. The principles of the behavior of the substituents apply as well to Reactions Schemes 4-6.

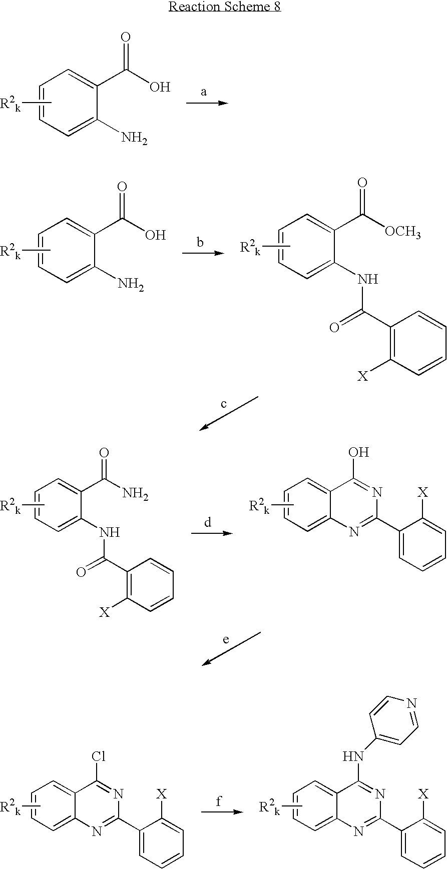

Reaction Scheme 8 shows a modified form of Reaction Scheme 3 which includes substituents R² in the quinazoline ring of formula (1). The substituents are carried throughout the reaction scheme. In step a, the starting material is treated with thionyl chloride in the presence of methanol and refluxed for 12 hours. In step b, the appropriate substituted benzoyl chloride is reacted with the product of step a by treating with the appropriately substituted benzoyl chloride in pyridine for 24 hours. In embodiments wherein X (shown illustratively in the ortho-position) is fluoro, 2-fluorobenzoyl chloride is used as a reagent; where X is (for illustration ortho-chloro), 2-chlorobenzoyl chloride is used.

In step C, the ester is converted to the amide by treating in ammonium hydroxide in an aprotic solvent such as dimethyl formamide (DMF) for 24 hours. The product is then cyclized in step d by treatment with 10 N NaOH in ethanol and refluxed for 3 hours.

The resulting cyclized form is then converted to the chloride in step e by treating with thionyl chloride in chloroform in the presence of a catalytic amount of DMF under reflux for 4 hours. Finally, the illustrated 4-pyridylamino compound is obtained in step f by treating with 4-amino pyridine in the presence of potassium carbonate and DMF and refluxed for 2 hours.

In illustrative embodiments of Reaction Scheme 8, R² may, for example, provide two methoxy substituents so that the starting material is 2-amino-4,5-dimethoxy benzoic acid and the product is, for example, 2-(2-chlorophenyl)-4-(4-pyridylamino)-6,7-dimethoxyquinazoline.

In another illustrative embodiment, R² provides a single nitro; the starting material is thus, for example, 2-amino-5-nitrobenzoic acid and the resulting compound is, for example, 2(2-fluorophenyl)-4-(4-pyridylamino)-5-nitroquinazoline.

Reaction Schemes 4-6 can be carried out in a manner similar to that set forth in Reaction Scheme 8, thus carrying along R² substituents through the steps of the process.

In compounds of the invention wherein R² is nitro, the nitro group may be reduced to amino and further derivatized as indicated in Reaction Scheme 9.

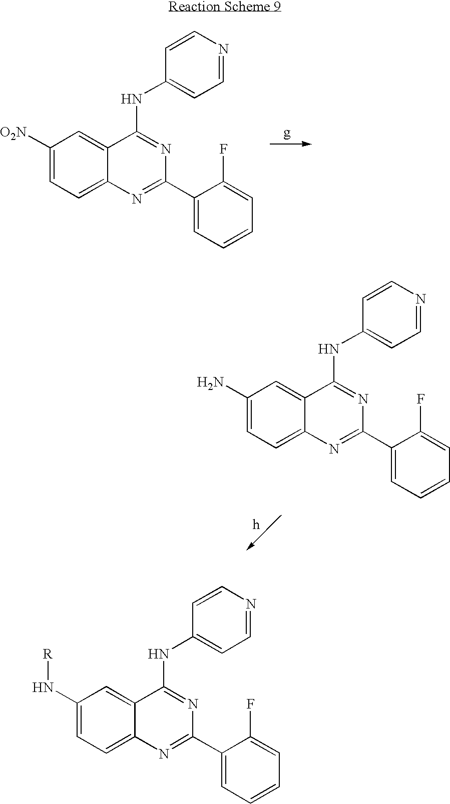

In Reaction Scheme 9, the illustrative product of Reaction Scheme 8 is first reduced in step g by treating with hydrogen and palladium on carbon (10%) in the presence of acetic acid and methanol at atmospheric pressure for 12 hours to obtain the amino compound. The resulting amino compound is either converted to the acyl form (R=acyl) using the appropriate acid chloride in the presence of chloroform and pyridine for four hours, or is converted to the corresponding alkylated amine (R=alkyl) by treating the amine intermediate with the appropriate aldehyde in the presence of ethanol, acetic acid, and sodium triacetoxyborohydride for 4 hours.

While the foregoing exemplary Reaction Schemes are set forth to illustrate the synthetic methods of the invention, it is understood that the substituents shown on the quinazoline ring of the products are generically of the formula (1) as described herein and that the reactants may be substituted accordingly. Variations to accommodate various substituents which represent embodiments of R³ other than the moieties shown in these illustrative examples or as Ar′ in these illustrative examples may also be used. Similarly, embodiments wherein the substituent at position 4 contains an arylalkyl can be used in these schemes. Methods to synthesize the compounds of the invention are, in general, known in the art.

Small organic molecules other than quinazoline derivatives can be synthesized by well known methods of organic chemistry as described in standard textbooks.

Compounds of formula (4) or (5) can be synthesized by methods well known in the art that will be readily apparent for those skilled in the art.

Methods of Treatment

The manner of administration and formulation of the compounds useful in the invention and their related compounds will depend on the nature and severity of the condition, the particular subject to be treated, and the judgment of the practitioner. The particular formulation will also depend on the mode of administration.

Thus, the small molecule compounds of the invention are conveniently administered by oral administration by compounding them with suitable pharmaceutical excipients so as to provide tablets, capsules, syrups, and the like. Suitable formulations for oral administration may also include minor components such as buffers, flavoring agents and the like. Typically, the amount of active ingredient in the formulations will be in the range of about 5%-95% of the total formulation, but wide variation is permitted depending on the carrier. Suitable carriers include sucrose, pectin, magnesium stearate, lactose, peanut oil, olive oil, water, and the like.

The compounds useful in the invention may also be administered through suppositories or other transmucosal vehicles. Typically, such formulations will include excipients that facilitate the passage of the compound through the mucosa such as pharmaceutically acceptable detergents.

The compounds may further be administered by injection, including intravenous, intramuscular, subcutaneous, intraarticular or intraperitoneal injection. Typical formulations for such use are liquid formulations in isotonic vehicles such as Hank's solution or Ringer's solution.

Alternative formulations include aerosol inhalants, nasal sprays, liposomal formulations, slow-release formulations, and the like, as are known in the art.

Any suitable formulation may be used.

If the compounds of the invention are used to counteract loss in β-adrenergic sensitivity resulting from the long-term or excessive use of another therapeutic agent, such as a β2-adrenergic agonist, their route of administration may also depend on the way the other therapeutic agent is administered. For example, β2-agonists used for the treatment of asthma, COPD and other diseases benefiting from the improvement of lung function (in particular from bronchodilation) are often administered as aerosol formulations for inhalation use. Concurrent administration of the compounds of the invention may, therefore, be conveniently performed by using the inhalation route, using the same or different formulation. The compounds of the invention may also be administered in combination with other therapeutic agents, such as natural or synthetic corticosteroids, particularly prednisone and its derivatives, and medications used in the treatment of cardiac diseases, such as congestive heart failure, including, without limitation, brain-derived natriuretic peptide (NBP).

A compendium of art-known formulations is found in Remington's Pharmaceutical Sciences, latest edition, Mack Publishing Company, Easton, Pa. Reference to this manual is routine in the art.

The dosages of the compounds of the invention will depend on a number of factors which will vary from patient to patient. However, it is believed that generally, the daily oral dosage will utilize 0.001-100 mg/kg total body weight, preferably from 0.01-50 mg/kg and more preferably about 0.01 mg/kg-10 mg/kg. The dose regimen will vary, however, depending on the conditions being treated and the judgment of the practitioner.

As implicated above, although the compounds of the invention may be used in humans, they are also available for veterinary use in treating non-human mammalian subjects.

Further details of the invention are illustrated by the following non-limiting example.

### EXAMPLE

Cell Preparations

Normal rat kidney cells (NRK) were cultured in DMEM-21 (high glucose)/10% FCS at 37° C., 5% CO2. Cells were serum starved for 24 hr before treated with 5 ng/ml huTGF-b1 (R&D System) or co-treatment with 1 μM Compound No. 81 or 0.1 μM Compound No. 74 for 24 hours.

Rat lung fibroblasts (RLF) were isolated from perfused rat lung by physical and enzymatic dissociation of lung tissue. Immunocytochemistry revealed most of the cells as fibroblasts. RLFs were cultured in FGM-2 with 2% FBS (Clonetics # CC-3132). Cells were serum starved for 24 hours, followed by the treatment with 15 ng/ml TGFβ1 (R&D System) or co-treatment with 1 μM Compound No. 81 or 0.1 μM Compound No. 74 for 24 hours.

Human lung fibroblasts (Cambrex Bio Science) from a 40 year old female were seeded at 4×10⁵ cells (passage 4) in 100 mm dishes and cultured in complete FGM medium (Cambrex Bio Science). The next day, medium was changed to FGM without serum or fibroblast growth factor, but supplemented with 0.2% bovine serum albumin and 50 μg/m; Vitamin C. Cells were serum deprived for 24 hours prior to treating with 5 ng/ml TGFβ1 (R&D Systems) in the presence or absence of 400 nM Compound No. 79. Induction with TGFβ was carried out for various times (7.5 hours, 24 hours, and 3 days) after which RNA was harvested by lysing the cells in RLT buffer (Qiagen) and frozen at −80° C.

cDNA Microarray

Gene expression profiles were determined from cDNA microarrays containing approximately 9000 elements derived from clones isolated from normalized cDNA libraries or purchased from ResGen (Invitrogen Life Technologies, Carlsbad, Calif.). DNA for spotting was generated by PCR amplification using 5′amino-modified primers (BD Biosciences Clontech, Palo Alto, Calif.) derived from flanking vector sequences. Amplified DNA was purified in a 96-well format using Qiagen's Qiaquick columns (Valencia, Calif.) according to the manufacturer's recommendations. Samples were eluted in Milli-Q purified water, dried to completion and resuspended in 7 μl of 3×SSC. A fluorescent assay using PicoGreen (Molecular Probes, Eugene, Oreg.) was randomly performed on 12% of the PCR products to determine the average yield after purification; yields were ˜1.5 μg of DNA which corresponds to a concentration of 214 μg/ml. Purified DNA was arrayed from 384-well microtiter plates onto lysine-coated glass slides using an OmniGrid II microarrayer (GeneMachines, San Carlos, Calif.). After printing, DNA was cross-linked to the glass with 65 mjoules UV irradiation and reactive amines were blocked by treatment with succinic anhydride For further details see, e.g. Eisen and Brown, Methods Enzymol. 303:179-205 (1999).

mRNA Isolation, Labeling and Hybridizations

Total RNA was extracted from cells using Qiagen's RNeasy kit. RNA was amplified using a modified Eberwine protocol (Eberwine et al., Proc. Natl. Acad. Sci. USA 89:3010-4 (1992)) that incorporated a polyA tail into the amplified RNA. Fluorescently-labeled cDNA probes were generated by reverse transcription of 4 μg of RNA with SuperScript II (Invitrogen Life Technologies, Carlsbad, Calif.) using anchored dT primers in the presence of Cy3 or Cy5 dUTP (Amersham, Piscataway, N.J.). Labeled cDNA probe pairs were precipitated with ethanol and purified using Qiaquick columns. Twenty μg each of poly(A) DNA, yeast tRNA, and human Cot1 DNA (Applied Genetics, Melbourne, Fla.) was added to the eluant. The samples were dried to completion and resuspended in 12.5 μl 3×SSC, 0.1% SDS. Probes were heated to 95° C. for 5 minutes, applied to the arrays under a 22 mm² cover slip and allowed to hybridize for at least 16 h at 65° C. The arrays were washed at 55° C. for 10 minutes in 2×SSC, 0.1% SDS, followed by two washes at room temperature in 1×SSC (10 min) and 0.2×SSC (15 min). Hybridization of each fluorophore was quantified using an Axon GenePix 4000A scanner.

Microarray Data Analysis

Differential expression values were expressed as the ratio of the median of background-subtracted fluorescent intensity of the experimental RNA to the median of background-subtracted fluorescent intensity of the control RNA. For ratios greater than or equal to 1.0, the ratio was expressed as a positive value. For ratios less than 1.0, the ratio was expressed as the negative reciprocal (i.e., a ratio of 0.5=−2.0). Median ratios were normalized to 1.0 using two pools of 3000 randomly chosen cDNAs in each pool. Six replicates of each of the two pools were printed in 4 evenly distributed blocks of the array. Expression data was rejected if neither channel produced a signal of at least 2.0-fold over background. Differential expression ratios were determined as the mean of the two values from dye-swapped duplicates. (Kerr and Churchill, Genet Res. 77:123-8 (2001).)

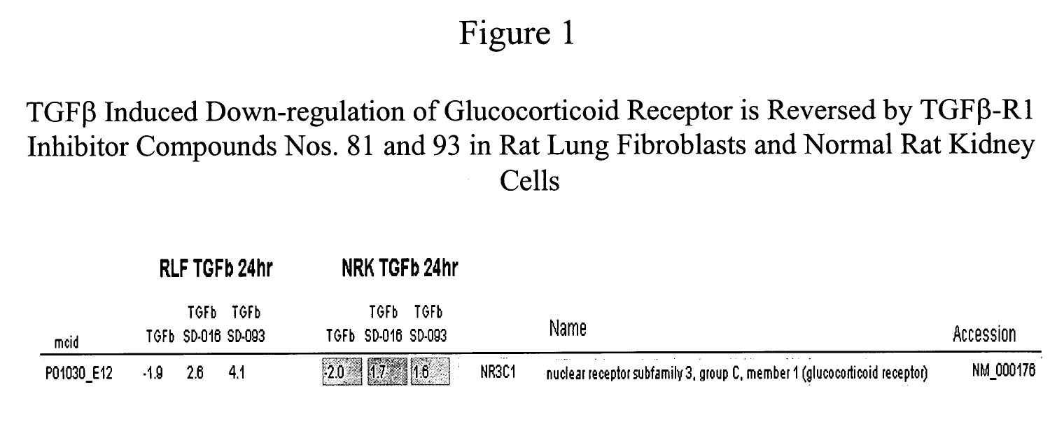

As shown in FIG. 1, TGF-β1 down-regulates glucocorticoid receptor expression in rat kidneys and lung fibroblast cells at the transcriptional level (2-fold). The down-regulation of glucocorticoid receptor expression is reversed by treatment with two representative TGFβ-R1 inhibitors designated Compounds Nos. 74 and 81.

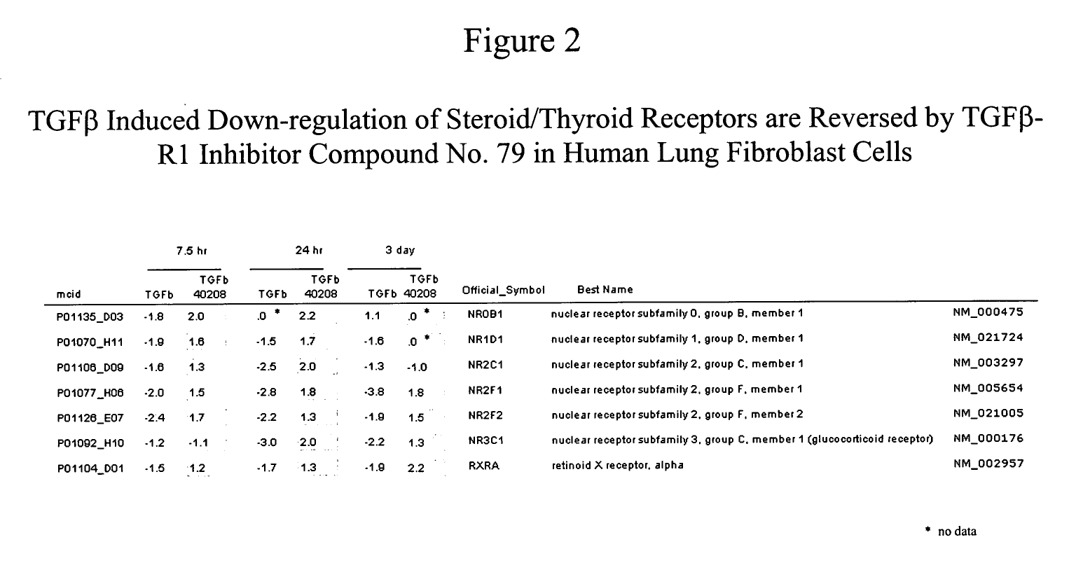

Separate microarray analysis to different human cDNA clones showed that TGF-β1 down-regulates seven members of the steroid nuclear receptor family, including the glucocorticoid receptor and retinoid receptor C alpha (FIG. 2). The down-regulation of these steroid/retinoid receptors was reversed by treatment with the TGFβ-R1 inhibitor Compound No. 79.

What is claimed is:

1. A method for counteracting a pathologic change in a signal-transduction pathway involving a member of the steroid/thyroid hormone super-family, comprising administering to a mammalian subject in need an effective amount of a compound capable of inhibiting TGF-β signaling through a TGF-β receptor.

2. The method of claim 1 wherein the receptor is a steroid hormone receptor.

3. The method of claim 2 wherein the pathologic change is down- or up-regulation of the steroid hormone receptor.

4. The method of claim 3 wherein the down- or up-regulation involves TGF-β.

5. The method of claim 3 wherein the down- or up-regulation is induced by TGF-β.

6. The method of claim 1 wherein the pathologic change is a TGF-β induced change in the activity or signaling of a steroid hormone receptor.

7. The method of claim 2 wherein the steroid hormone receptor is glucocorticoid receptor.

8. The method of claim 1 wherein the receptor is a thyroid hormone receptor.

9. The method of claim 8 wherein the pathologic change is down- or up-regulation of a thyroid hormone receptor.

10. The method of claim 9 wherein the down- or up-regulation involves TGF-β.

11. The method of claim 9 wherein the down- or up-regulation is induced by TGF-β.

12. The method of claim 8 wherein the pathologic change is a TGF-β induced change in the activity or signaling of a thyroid hormone receptor.

13. The method of claim 1 wherein the receptor is a retinoic acid receptor.

14. The method of claim 13 wherein the pathologic change is down- or up-regulation of a retinoic acid receptor.

15. The method of claim 14 wherein the down- or up-regulation involves TGF-β.

16. The method of claim 14 wherein the down- or up-regulation is induced by TGF-β.

17. The method of claim 13 wherein the pathologic change is a TGF-β induced change in the activity or signaling of a retinoic acid receptor.

18. The method of claim 1 wherein the TGF-β receptor is a TGFβ-R1 kinase.

19. The method of claim 18 wherein the compound is capable of binding to said TGFβ-R1 kinase.

20. The method of claim 19 wherein the compound is capable of binding to an additional receptor kinase.

21. The method of claim 20 wherein the additional receptor kinase is an activin receptor (Alk4).

22. The method of claim 1 wherein the compound is a non-peptide small molecule.

23. The method of claim 22 wherein the compound is a small organic molecule.

24. The method of claim 23 wherein the small organic molecule is a compound of formula (1) 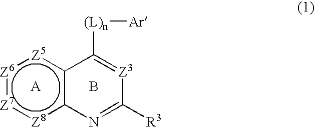
or the pharmaceutically acceptable salts thereof
wherein R³ is a noninterfering substituent;
each Z is CR² or N, wherein no more than two Z positions in ring A are N, and
wherein two adjacent Z positions in ring A cannot be N;
each R² is independently a noninterfering substituent;
L is a linker;
n is 0 or 1; and
Ar′ is the residue of a cyclic aliphatic, cyclic heteroaliphatic, aromatic or heteroaromatic moiety optionally substituted with 1-3 noninterfering substituents.

25. The method of claim 24 wherein the compound is a quinazoline derivative.

26. The method of claim 25 wherein Z³ is N; and Z⁵-Z⁸ are CR².

27. The method of claim 25 wherein Z³ is N; and at least one of Z⁵-Z⁸ is nitrogen.

28. The method of claim 25 wherein R³ is an optionally substituted phenyl moiety.

29. The method of claim 28 wherein R³ is selected from the group consisting of 2-4-, 5-, 2,4- and 2,5-substituted phenyl moieties.

30. The method of claim 29 wherein at least one substituent of the phenyl moiety is an alkyl(1-6C), or halo.

31. The method of claim 23, wherein the small organic molecule is a compound of formula (2) 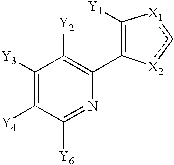
wherein Y₁ is phenyl or naphthyl optionally substituted with one or more substituents selected from halo, alkoxy(1-6 C), alkylthio(1-6 C), alkyl(1-6 C), haloalkyl (1-6C), —O—(CH₂)ₘ-Ph, —S—(CH₂)ₘ-Ph, cyano, phenyl, and CO₂R, wherein R is hydrogen or alkyl(1-6 C), and m is 0-3; or phenyl fused with a 5- or 7-membered aromatic or non-aromatic ring wherein said ring contains up to three heteroatoms, independently selected from N, O, and
Y₂, Y₃, Y₄, and Y₅ independently represent hydrogen, alkyl(1-6C), alkoxy(1-6 C), haloalkyl(1-6 C), halo, NH₂, NH-alkyl(1-6C), or NH(CH₂)ₙ-Ph wherein n is 0-3; or an adjacent pair of Y₂, Y₃, Y₄, and Y₅ form a fused 6-membered aromatic ring optionally containing up to 2 nitrogen atoms, said ring being optionally substituted by one or more substituents independently selected from alkyl(1-6 C), alkoxy(a-6 C), haloalkyl(1-6 C), halo, NH₂, NH-alkyl(1-6 C), or NH(CH₂)ₙ-Ph, wherein n is 0-3, and the remainder of Y₂, Y₃, Y₄, and Y₅ represent hydrogen, alkyl(1-6 C), alkoxy(1-6C), haloalkyl(1-6 C), halo, NH₂, NH-alkyl(1-6 C), or NH(CH₂)ₙ-Ph wherein n is 0-3; and
one of X₁ and X₂ is N and the other is NR₆, wherein R₆ is hydrogen or alkyl(1-6C).

32. The method of claim 23 wherein the small organic molecule is a compound of formula (3) 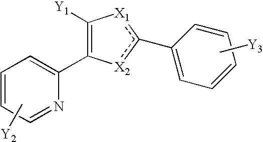
wherein Y₁ is naphthyl, anthracenyl, or phenyl optionally substituted with one or more substituents selected from the group consisting of halo, alkoxy(1-6 C), alkylthio(1-6 C), alkyl(1-6 C), —O—(CH₂)-Ph, —S—(CH₂)ₙ-Ph, cyano, phenyl, and CO₂R, wherein R is hydrogen or alkyl(1-6 C), and n is 0, 1, 2, or 3; or Y₁ represents phenyl fused with an aromatic or non-aromatic cyclic ring of 5-7 members wherein said cyclic ring optionally contains up to two heteroatoms, independently selected from N, O, and S;
Y₂ is H, NH(CH₂)ₙ-Ph or NH-alkyl(1-6 C), wherein n is 0, 1, 2, or 3;
Y₃ is CO₂H, CONH₂, CN, NO₂, alkylthio(1-6 C), —SO₂-alkyl(C1-6), alkoxy(C1-6), SONH2, CONHOH, NH₂, CHO, CH₂NH₂, or CO₂R, wherein R is hydrogen or alkyl(1-6 C);
one of X₁ and X₂ is N or CR′, and other is NR′ or CHR′ wherein R′ is hydrogen, OH, alkyl(C-16), or cycloalkyl(C3-7); or when one of X₁ and X₂ is N or CR′ then the other may be S or O.

33. The method of claim 23 wherein the small organic molecule is a compound of formula (4) 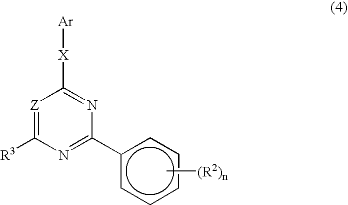
and the pharmaceutically acceptable salts and prodrug forms thereof; wherein
Ar represents an optionally substituted aromatic or optionally substituted heteroaromatic moiety containing 5-12 ring members wherein said heteroaromatic moiety contains one or more O, S, and/or N with a proviso that the optionally substituted Ar is not 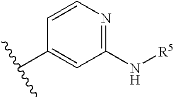
wherein R⁵ is H, alkyl (1-6C), alkenyl (2-6C), alkynyl (2-6C), an aromatic or heteroaromatic moiety containing 5-11 ring members;
X is NR¹, O, or S;
R¹ is H, alkyl (1-8C), alkenyl (2-8C), or alkynyl (2-8C);
Z represents N or CR⁴;
each of R³ and R⁴ is independently H, or a non-interfering substituent;
each R² is independently a non-interfering substituent; and
n is 0, 1, 2, 3, 4, or 5. In one embodiment, if n>2, and the R²'s are adjacent, they can be joined together to form a 5 to 7 membered non-aromatic, heteroaromatic, or aromatic ring containing 1 to 3 heteroatoms where each heteroatom can independently be O, N, or S.

34. The method of claim 23 wherein the small organic molecule is a compound of formula (5) 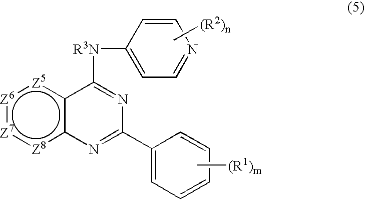
or the pharmaceutically acceptable salts thereof;
wherein each of Z⁵, Z⁶, Z⁷ and Z8 is N or CH and wherein one or two Z⁵, Z⁶, Z⁷ and Z⁸ are N and wherein two adjacent Z positions cannot be N;
wherein m and n are each independently 0-3;
wherein two adjacent R′ groups may be joined to form an aliphatic heterocyclic ring of 5-6 members;
wherein R² is a noninterfering substituent; and
wherein R³ is H or CH₃.
